(19) United States
(12) Patent Application Publication
(10) Pub. No.: US 2026/0028349 A1
(43) Pub. Date: Jan. 29, 2026

(54) DIMERIC FORM OF BENZOPORPHYRIN DERIVATIVE PHOTOSENSITIZER AND NANOPARTICLES AND METHODS THEREOF

(71) Applicant: University of Maryland, College Park, College Park, MD (US)

(72) Inventors: Huang-Chiao Huang, College Park, MD (US); Carla Arnau del Valle, College Park, MD (US)

(21) Appl. No.: 19/282,960

(22) Filed: Jul. 28, 2025

Related U.S. Application Data
(60) Provisional application No. 63/676,073, filed on Jul. 26, 2024.

(51) Int. Cl. C07D 487/22; A61K 9/51; A61K 41/00; A61P 35/00

(52) U.S. Cl. CPC C07D 487/22; A61K 9/5146; A61K 41/008; A61P 35/00

(57) ABSTRACT

Photodynamic therapy (PDT) is a minimally invasive treatment that involves the administration of a light-activatable drug followed by light activation of the lesion to produce reactive oxygen species that kill cancer cells. VISUDYNE®, a liposomal formulation of benzoporphyrin derivative (BPD) photosensitizer, is clinically approved for PDT of ocular diseases and is now being tested for PDT and imaging of pancreatic, brain, and other cancers. While VISUDYNE® improves the pharmacokinetics of BPD, it lacks treatment selectivity. This present disclosure is directed to dBPD, dBPD nanoparticles, and preparation and use thereof that provide cancer treatment selectivity for cancers characterized by overexpression of folate receptor (FR).

### CROSS REFERENCE TO RELATED APPLICATIONS

This application claims priority to U.S. Application No. 63/676,073, filed on Jul. 26, 2024, the contents of which are hereby incorporated by reference in its entirety.

### STATEMENT OF FEDERALLY SPONSORED RESEARCH AND DEVELOPMENT

### Field of the Invention

The field of the invention relates generally to the composition and method of synthesizing and using dimeric benzoporphyrin derivative (dBPD) photosensitizer and dBPD-loaded nanoparticles (dBPD-NPs).

### BACKGROUND

This background information is provided for the purpose of making information believed by the applicant to be of possible relevance to the present invention. No admission is necessarily intended, nor should it be construed, that any of the information disclosed herein constitutes prior art against the present invention.

Ovarian cancer is the fifth most frequent cause of death in women, claiming the highest mortality rate within gynecological cancers. The standard of care for advanced-stage ovarian cancer is surgical debulking and platinum-based chemotherapy, followed by maintenance treatment with PARP inhibitors and/or bevacizumab. Despite these, the majority of women face relapse, resulting in a concerning 5-year survival rate of approximately 30%. To mitigate treatment-associated toxicity while augmenting therapeutic outcomes, targeted therapies emerge as promising avenues. Folate receptor (FR) is overexpressed in over 70% of primary and 80% of recurrent ovarian cancers. Additionally, the expression of FR has been shown to remain high after chemotherapy. Thus, there is an urgent need for more effective and targeted treatment strategies that can overcome the limitations of clinical drugs.

Photodynamic therapy (PDT) is a minimally invasive treatment in which administration of a light-activated drug (photosensitizers) is followed by irradiation at a specific wavelength leading to the production of cytotoxic reactive oxygen species. Most of the photosensitizers present a limited clinical application due to their poor water solubility and limited selectivity to cancer cells. One of the prominent photosensitizers, benzoporphyrin derivative (BPD), has demonstrated great results in preclinical and clinical studies due to its high phototoxicity in different cancers. However, BPD, like many other photosensitizers, shows poor water solubility and limited tumor selectivity, which can lead to off-target effects and reduced therapeutic efficacy.

To address these challenges, BPD is clinically delivered as a liposomal formulation, VISUDYNE®, which is currently used for the treatment of age-related macular degeneration. The use of nanoparticles offers a versatile platform that can be engineered with drugs, like photosensitizers, as well as functionalized with different targeting moieties, yielding a biocompatible nanoformulation for targeted drug delivery therapies. Among these, lipid-based nanoparticles, like liposomes, micelles, solid-lipid nanoparticles (SLNPs), etc. have shown great results due to their excellent biocompatibility, long circulation times and high tumor accumulation via the enhanced permeability and retention (EPR) effect.

Lipid-based nanoparticles, particularly PEGylated lipid nanoassemblies, have emerged as a superior approach for the delivery of hydrophobic photosensitizers. These nanoassemblies are synthesized through the self-assembly of the hydrophobic drugs and encapsulation in PEG-lipid monomers, such as DSPE-PEG. The hydrophobic DSPE lipid tail drives strong self-assembly, while the PEG headgroup provides the necessary curvature to form stable oblate spheroidal micelles. This design allows for the efficient encapsulation of hydrophobic drugs, enhancing their solubility and stability. Despite many advantages offered by the nanoformulations of photosensitizers for PDT, several limitations greatly hinder the clinical translation of these nanomedicines, mainly due to their low drug loading efficiency, poor colloidal stability and potential premature drug leakage.

One strategy to enhance the drug loading efficiency and nanoparticle stability and thus, the therapeutic efficacy is the use of dimeric formulations of the corresponding photosensitizers. Conjugating two hydrophobic drug molecules can significantly increase their overall hydrophobicity and size compared to their monomeric forms, facilitating better encapsulation within nanoparticles and enhancing their self-assembly effect. Additionally, these dimers can also form nanoassemblies via intermolecular hydrophobic forces and π-π stacking effect. Dimeric-based nanosystems are typically synthesized via a chemical linkage and researchers have leveraged the use of tumor stimuli-responsive linkers to further control the drug release.

In the context of ovarian cancer, glutathione (GSH) levels are often elevated within tumor cells compared to healthy cells. Additionally, the intracellular concentration of GSH is found to be significantly higher (2-20 mM) than that in the extracellular microenvironment (2-20 μM).

Leveraging these higher GSH levels present in cancer cells, redox-responsive linkages have been extensively explored. In particular, disulfide-containing linkages have been widely used as the gold standard for designing GSH-responsive drug delivery systems. For instance, researchers have synthesized dimeric compounds based on chemotherapeutic drugs such as doxorubicin and paclitaxel and photosensitizers such as boron dipyrromethene (BODIPY) derivatives, or combinations of chemo-drugs and photosensitizers or different phototherapies. The disulfide-induced nanoassembly or nanoparticle formation strategy has been used to encapsulate dimers in nanosystems such as liposomes, micelles, and polymeric nanoparticles, as well as for self-assembled nanoparticles.

The combination of GSH-targeting and PDT is also particularly compelling because GSH can consume reactive oxygen species (ROS) generated during PDT. By depleting GSH through GSH-triggered drug release, the ability of the cancer cells to neutralize ROS is diminished, thereby enhancing the effectiveness of the PDT and leading to enhanced killing of cancer cells.

Folate receptors are overexpressed in ovarian cancer cells, making them an attractive target for enhancing the specificity of the drug delivery. These receptors have a high affinity for folic acid, which can be utilized to decorate nanoparticles for the targeting capabilities enhancing their uptake by ovarian cancer cells. Leveraging both the GSH-triggered drug release and the folate receptor-mediated targeting could provide an enhanced accumulation of the photosensitizer into cancer cells as well as an improved PDT effect and reduced side effects to healthy tissues.

### SUMMARY

One aspect of the invention pertains to a dimeric benzoporphyrin derivative (dBPD) of Formula I:

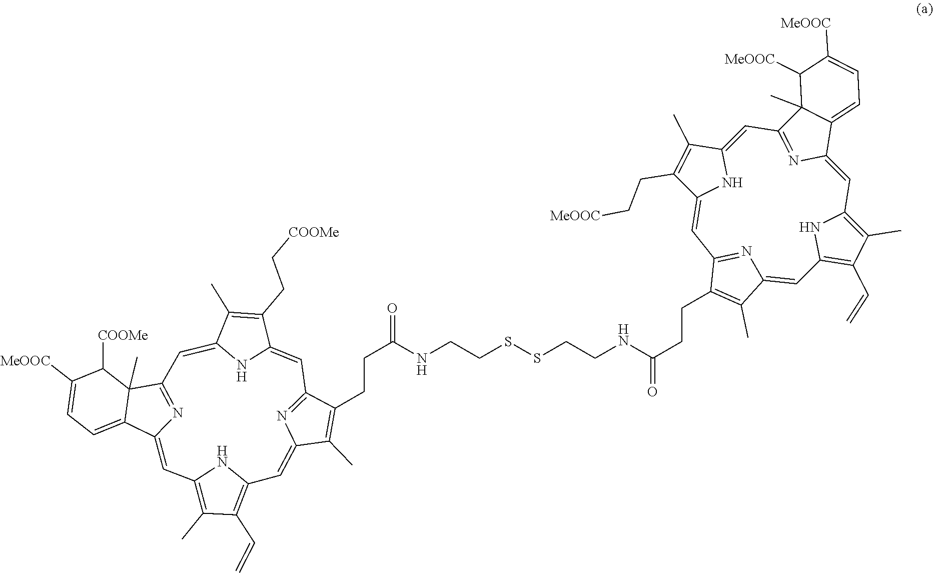 [SMILES: C=Cc1c(C)c2cc3nc(cc4[nH]c(cc5nc(cc1[nH]2)C(C)=C5CCC(=O)NCCSSCCNC(=O)CCC1=C(C)c2cc5[nH]c(cc6nc(cc7[nH]c(cc1n2)c(CCC(=O)OC)c7C)C1(C)C6=CC=C(C(=O)OC)C1C(=O)OC)c(C)c5C=C)c(CCC(=O)OC)c4C)C1(C)C3=CC=C(C(=O)OC)C1C(=O)OC]

The dimeric molecule, Formula I, is hydrophobic and is formed by conjugating two benzoporphyrin derivative molecules using a cystamine as the linker using a EDC/HOBt coupling reaction between amine groups in the cystamine and the carboxyl group on BPD.

A further aspect of the inventions pertains to a nanoparticle composition comprising dBPD of Formula I, and polyethylene glycol (PEG). The nanoparticle composition is in the form of a dBPD-loaded nanoparticle as illustrated in FIG. 3a. Ther nanoparticle composition is in the form of a liposome and the dBPD is encapsulated within the nanoparticle.

A further aspect of the invention pertains to a nanoparticle composition comprising dBPD of Formula I, polyethylene glycol (PEG), and folic acid. The nanoparticle composition is in the form of a dBPD-loaded nanoparticle as illustrated in FIG. 3a. Ther nanoparticle composition is in the form of a liposome and the dBPD is encapsulated within the nanoparticle and the folic acid is tethered to the PEG on the outer portion of the capsule, as illustrated in FIG. 23. The folic acid is tethered to the PEG and is present on at least 50% or more of the surface of the nanoparticle.

The nanoparticle composition is photoactive, and the photoactivated nanoparticles are selectively cytotoxic to cancer cells only, and not cytotoxic to healthy cells.

A further aspect of the invention pertains to a formulation comprising a nanoparticle composition as described in some embodiments, and one or more pharmaceutically acceptable excipient, wherein the pharmaceutically acceptable excipient is water. The formulation is administered parenterally or intravenously to a patient or a subject in need thereof. The formulation is photoactive, and the photoactivated formulation is selectively cytotoxic to cancer cells only, and selectively delivered to the endoplasmic reticulum of the cancer cells. The formulation is not cytotoxic to healthy cells.

A further aspect of the invention pertains to a method of making a dimeric molecule. The method comprises linking two BPD moieties. A stock solution of cystamine was prepared by dissolving it (6.4 mg, 42.03 μmol) in dimethylformamide (DMF) (8.5 mL). N,N-Diisopropylethylamine (DIPEA) (2 mL, 11.48 mmol) was added to the previous mixture and stirred for 15 min. Next, the previous stock solution (4.5 mL, 18.01 μmol of cystamine) was added to a vial containing 1-Ethyl-3-(3-dimethylaminopropyl) carbodiimide (EDC) (60 mg, 312.99 μmol), 1-Hydroxybenzotriazole (HOBt) (29.1 mg, 215.36 μmol) and benzoporphyrin derivative (BPD, US Pharmacopeia) (26.5 mg, 36.87 μmol) in DMF (1.5 mL). The reaction mixture was left stirring at 35° C. for 48 h. After this time, the product was extracted using diethyl ether/H2O mixture and the organic phase was washed 3 times with H2O to remove the excess DMF and the final product was dried under vacuum. To further purify the dBPD, the product was dissolved in dichloromethane (DCM) (2 mL) and loaded into a preparative TLC plate (TLC Silica Gel 60 F254 Multiformat, Millipore Sigma) run using ethyl acetate. The product was extracted from the TLC and separated from the silica by extraction with diethyl ether, followed by 5 washes centrifugating at 1000 rpm for 5 min. After evaporation of the solvent, dBPD was obtained as a dark green powder (14.6 mg, 51%). 1H (600 MHz, DMSO-d6, δ): 9.90 (1H, s), 9.83-9.81 (1H, m), 9.73-9.61 (5H, m), 9.33-9.28 (2H, m), 8.43-8.38 (1H, m), 8.32-8.16 (1H, m), 8.06-8.02 (1H, m), 7.81-7.77 (5H, m), 6.50-6.04 (5H, m), 5.27-5.25 (2H, m), 4.30-4.06 (8H, m), 3.93-3.91 (10H, m), 3.63 (2H, s), 3.58 (4H, s), 3.54-3.44 (15H, m), 2.96-2.93 (9H, m), 2.90 (10H, s), 2.74 (4H, s) (Figure S1). MS-MALDI was obtained in linear/positive mode using DHB as the matrix in a Bruker MALDI-TOF (Figure S2). HRMS (ESI) m/z: [M+H]+ calcd for C86H92N10O14S2 1553.6309. found, 1553.5861. The presence of different isomers was demonstrated by liquid chromatography-electrospray ionization mass spectrometry (LC-ESI+) as shown in Figure S3. The absorption and the fluorescence emission spectra of the dBPD were recorded (Cytation 5, BioTek) in dimethyl sulfoxide (DMSO) and with different amounts of H2O (0, 20, 40, 60, 80 and 98%).

A further aspect of the invention pertains to a method of making a dBPD-loaded nanoparticle, said method comprising dissolving dBPD in an organic polar colvent, mixing the solution with a pegylated lipid, evaporating the organic solvent from the mixture to form a solid, hydrating the solid with an aqueous solution, and freeze thawing the solution to for dBPD-NPs.

A further aspect of the invention pertains to a method of making a folic acid functionalized dBPD-loaded nanoparticle said method comprising dissolving dBPD in an organic polar colvent, mixing the solution with a pegylated lipid, adding folic acid to the mixture, evaporating the organic solvent from the mixture to form a solid, hydrating the solid with an aqueous solution, and freeze thawing the solution to for dBPD-NPs.

A further aspect of the invention pertains to treating cancer comprising administering a pharmaceutically effective amount of dBPD or formulation to a patient of subject in need thereof. The cancer is a cancer that involves the overexpression of folate receptors such as lung cancer, ovarian cancer, endometrial cancer or breast cancer.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Synthesis and purification of dBPD. Chemical reaction of dBPD with cystamine to yield dBPD.

FIG. 2. Characterization of dBPD. FIG. 2a. Normalized UV-VIS absorption spectra of BPD and dBPD in DMSO. FIG. 2b. Normalized fluorescence emission spectra of BPD and dBPD in DMSO (λ_{exc}=450 nm). FIG. 2c. MS-MALDI spectrum of dBPD. FIG. 2d. LC-MS spectra of isomers form in the synthesis of dBPD. FIG. 2e. Fluorescence emission intensity of BPD and dBPD at different H₂O:DMSO ratios.

FIG. 3. Synthesis and optimization of dBPD-NPs. FIG. 3a. Schematic illustration of the synthesis of PEGlyated lipid nanoparticles encapsulating dBPD (dBPD-NPs). FIG. 3b. Representative intensity plot of dBPD-NPs (0.22 mg) recorded by DLS; characterization of dBPD-NPs. FIG. 3c. The hydrodynamic diameter (d). FIG. 3d. Polydispersity index (PdI). FIG. 3e. Zeta potential. FIG. 3f. Encapsulation efficiency (EE). FIG. 3g. Loading capacity (LC). FIG. 3h. Fluorescence emission quenching of dBPD.

FIG. 4. GSH-triggered BPD release. Comparative drug release profiles of dBPD-NPs (45 μM) in PBS at 4° C. (purple) and 37° C. with 0 (blue), 2 (green), and 20 mM (red) GSH. * p≤0.05; **** p≤0.0001.

FIG. 5. Optimization of folic acid on dBPD-NPs for FR targeting. Characterization of dBPD-NPs, 1% FA-dBPD-NPs and 10% FA-dBPD-NPs. FIG. 5a. The hydrodynamic diameter (d). FIG. 5b. Zeta potential. FIG. 5c. Polydispersity index (PdI). FIG. 5d. Encapsulation Efficiency (EE). FIG. 5e. Loading capacity (LC). FIG. 5f. Fluorescence emission quenching of dBPD. FIG. 5h. Representative digital image of 1% FA-dBPD-NPs in PBS. * p≤0.05; *** p≤0.001.

FIG. 6. Size and stability of dBPD-NPs. FIG. 6a. Cryogenic transmission election microscopy (cryo-TEM) images of the 1% FA-dBPD-NPs and the data distribution (261 NPs counted); over time stability of the NPs recording. FIG. 6b. The size. FIG. 6c. The PdI by DLS (dBPD-NPs in blue, 1% FA-dBPD-NPs in red and 10% FA-dBPD-NPs in green). Scale bar 0.5 μm and 100 nm.

FIG. 7. Photoactivity and singlet oxygen formation of the dBPD-NPs. Photoactivity values for dBPD, dBPD-NPs, 1% FA-dBPD-NPs and 10% FA-dBPD-NPs (2 M) in: FIG. 7a. 1% HAS. Normalized SOSG fluorescence emission wavelength intensity (λ_{exc}=504 nm/λₑₘ=525 nm) before and after irradiation at 689 nm (40 J/cm², 150 mW/cm²) of control, dBPD, dBPD-NPs, 1% FA-dBPD-NPs and 10% FA-dBPD-NPs in FIG. 7c. In PBS and FIG. 7d. In 1% HAS. * p≤0.05; ** p≤0.01; *** p≤0.001; **** p≤0.0001.

FIG. 8. Quantification of the dBPD uptake by cells and FA-targeting ability. Uptake of dBPD in FIG. 8a. OVCAR8, FIG. 8b. 3T3 cells treated with dBPD-NPs, 1% FA-dBPD-NPs and 10% FA-dBPD-NPs (2.5 μM, 24 h). FIG. 8c. Confocal images of fixed OVCAR8 cells incubated with dBPD-NPs or 1% FA-dBPD-NPs (2.5 μM, 24 h). Images recorded using 20×, Hoechst channel, (λ_{exc}=405 nm, λₑₘ=430-470 nm), dBPD channel (λ_{exc}=445 nm, λₑₘ=650-750 nm) and overlay of both channels. Scale bar 50 μm. n=3-6; **** p≤0.0001.

FIG. 9. Colocalization studies of dBPD and dBPD-NPs. Confocal images of live OVCAR8 cells incubated with dBPD or dBPD-NPs (2.5 μM, 24 h). Images recored using 60× Hoechst channel, (λ_{exc}=405 nm, λₑₘ=430-470 nm), dBPD channel (λ_{exc}=445 nm, λₑₘ=650 750 nm), MitoTracker-Green and ER-tracker Green channel (λ_{exc}=488 nm, λₑₘ=500-535 nm), and Lysotracket Red DND-99 channel (λ_{exc}=561 nm, λₑₘ=565-620 nm). Scale bar 20 μm.

FIG. 10. Analysis of the colocalization of dBPD-NPs in cancer cells. Colocalization plot intensity graphs of dBPD with: FIG. 10a. Mitotracker-Green, FIG. 10b. Lysotracker-Red, and FIG. 10c. ERtracker-Green; and of dBPD-NPs with: FIG. 10d. Mitotracker-Green, FIG. 10e. Lysotracker-Red, and FIG. 10f. ERtracker-Green. Insets are the corresponding confocal scanning microscopy images of OVCAR8 cells treated with dBPD or dBPD-NPs (2.5 μM, 24 h) and with the corresponding trackers. Spectra in green correspond to the Mitotracker-Green, spectra in red correspond to Lysotracker-Red, spectra in black correspond to ERtracker-green and spectra in purple correspond to the dBPD signals.

FIG. 11. dBPD-NPs for photodynamic therapy in cancer cells. FIG. 11a. OVCAR8 cell viability measured via CellTiter-Glo® Cell Viability Assay at 48 h after PDT using dBPD, dBPD-NPs, 1% FA-dBPD-NPs, 10% FA-dBPD-NPs or without treatment (690 nm, 0-50 J/cm², 150 mw/cm²); FIG. 11b. OVCAR8 spheroids viability measured via CellTiter-Glo® Cell Viability Assay at 48 h after PDT using dBPD-NPs, 1% FA-dBPD-NPs or without treatment (690 nm, 0-10 J/cm², 150 mw/cm²); and FIG. 11c. representative images of spheroids on day 7 at each light dose. Scale bar=100 μm.

FIG. 12. dBPD-NPs for photodynamic therapy in cancer mouse model. FIG. 12a. Biodistribution of dBPD-NPs and 1% FA-dBPD-NPs, FIG. 12b. tumor burden showing the tumor weights in grams of the different organs with no treatment (NT), with the 1% FA-dBPD-NPs at dark conditions and with 1% FA-dBPD-NPs at PDT 690 nm, 100 J/cm², 150 mw/cm² and FIG. 12c. total tumor burden in NT, dark and PDT conditions.

FIG. 13. FIG. 13a. Absorption and FIG. 13b. fluorescence spectra of dBPD, and FIG. 13c. absorption and FIG. 13d. fluorescence spectra of dBPD-NPs in PBS (blue) and DMSO (red).

FIG. 14. Fluorescence emission spectra of FIG. 14a. BPD (5 μM) and FIG. 14b. dBPD (5 μM) solutions containing different amounts of H₂O in DMSO; FIG. 14c. Corresponding fluorescence emission intensity at 700 nm of BPD (blue) and dBPD (red) at different amounts of H₂O in DMSO.

FIG. 15. Cryogenic transmission electron microscopy (cryo-TEM) of dBPD-NPs: FIG. 15a. data distribution (179 NPs counted), FIG. 15b. image of dBPD-NPs with scale bar 0.5 μm, and FIG. 15c. image of dBPD-NPs with scale bar 200 nm.

FIG. 16. FIG. 16a. Normalized UV-Vis absorption spectra and FIG. 16b. normalized fluorescence emission spectra of dBPD (blue), dBPD-NPs (red), 1% FA-dBPD-NPs (green) and 10% FA-dBPD-NPs (purple) in DMSO. λ_{exc}=450 nm.

FIG. 17. Results from the synthesis of dBPD-NPs, BPD-NPs and empty-NPs: FIG. 17a. Photo of the samples after the freeze-thaw protocol and before filtration; FIG. 17b. photo of the filters after samples filtration; and FIG. 17c. representative intensity plots of the samples post-dialysis recorded using dynamic light scattering (DLS).

FIG. 18. Intensity plots of the BPD-NPs were recorded using DLS.

FIG. 19. Cell viability of dBPD (blue) and dBPD-NPs (red); FIG. 19a. OVCAR8 and FIG. 19b. NCI/ADR-Res cells treated with various concentrations of the drugs for 24 h (n=3).

FIG. 20. Western blot image of FR expression in OVCAR8 3T3 cells.

FIG. 21. Representative confocal images of live OVCAR8 cells incubated with dBPD-NPS or 1% FA-dBPD-NPs (2.5 μM, 24 h). Images recorded using 60×, DAPI channel (λ_{exc}=405 nm, λₑₘ=430-470 nm), dBPD channel (λ_{exc}=445 nm, λₑₘ=650-750 nm), brightfield (TD recorded using λ_{exc}=445 nm) and overlay of all channels. Scale bar 30 μm.

FIG. 22. Schematic of mechanism of action of dBPD-NPs. The dBPD-NPs are internalized via folate receptor (FR)-mediated endocytosis (1), followed by intracellular release of BPD is response to glutathione (GSH) (2). The dBPD/BPD selectively accumulates in the endoplasmic reticulum (ER) (3). Upon irradiation at 690 nm and activation of dBPD/BPD, photodynamic therapy (PDT) leads to cancer cell death (4).

FIG. 23. Synthesis of FA-dBPD-NPs: Schematic illustration of the synthesis of PEGylated lipid nanoparticles with folic acid (FA)-modified PEGylated lipids and encapsulating dBPD (FA-dBPD-NPs).

### DESCRIPTION

The subject matter of the present disclosure relates generally to photosensitizers and nanoparticles. More particularly, the present disclosure relates to the composition and method of synthesizing and using dimeric benzoporphyrin derivative (dBPD) photosensitizer and dBPD-loaded nanoparticles (dBPD-NPs). Embodiments of the present disclosure include a method of synthesis of a dimeric form of benzoporphyrin derivative (dBPD). In another embodiment, dBPD-NPs are prepared for photodynamic therapy and imaging, and inhibition of YAP-TAZ function. In one particular embodiment of preparing dBPD-NPs, dBPD is dissolved in an organic solvent like chloroform and mixed with pegylated lipid. After solvent evaporation, the solid formed is hydrated with aqueous solution and dBPD-NPs are formed following a free-thaw protocol. The dBPD-NPs are filtered and dyalised. In one or more disclosed embodiments, the dBPD-NPs size and dBPD product loading are tuneable. In yet another embodiment of the present disclosure, Folic acid (FA) functionalized nanoparticles (FA-dBPD-NPs) are prepared by incorporating FA in the synthesis of the nanoparticles before evaporation of the solvent. In at least one aspect of the disclosed embodiments, the number of FA molecules conjugates can be tuneable.

Herein, a self-assembled nanomedicine based on a dimeric form of BPD (dBPD-NPs) with a GSH-responsive linker for the photodynamic therapy of ovarian cancer was designed and developed. Engineered with folic acid, nanoparticles are further leveraged for the specific targeting of folate receptor-overexpressing ovarian cancer cells, offering a promising approach for improving PDT outcomes in ovarian cancer treatment.

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

A “therapeutically effective amount” as used herein refers to the amount of the nanoformulation sufficient to elicit a desired biological response in a subject, e.g., such as an amount sufficient to kill, reduce or stabilize cells associated with a disease or condition and/or sufficient to reduce symptoms associated with such disease or condition. Actual dosage levels of the active ingredient(s) in the disclosed formulations may be varied so as to obtain an amount of the active ingredient which is effective to achieve the desired therapeutic response for a particular patient, composition, and mode of administration. The formulations and compositions of the present invention may be administered by any suitable route and mode (e.g., parenteral, injected, topical, oral, intranasal, etc.).

As used herein, a “photosensitizer” or “photoreactive agent” refers to a compound or composition that is useful in photodynamic therapy in that it absorbs electromagnetic radiation and emits energy sufficient to exert a therapeutic effect, e.g., the impairment or destruction of unwanted cells or tissue, or sufficient to be detected in diagnostic applications. Photodynamic therapy according to the invention can be performed using any of a number of photoactive compounds. For example, the photosensitizer can be any chemical compound that collects in one or more types of selected target tissues and, when exposed to the light of a particular wavelength, absorbs the light and induces impairment or destruction of the target tissues. Virtually any chemical compound that homes to a selected target and absorbs light may be used in this invention. Preferably, the photosensitizer is nontoxic to the patient to which it is administered and is capable of being formulated in a nontoxic composition. The photosensitizer is also preferably nontoxic in its photodegraded form. Ideal photosensitizers are characterized by a lack of toxicity to cells in the absence of the photochemical effect and are readily cleared from non-target tissues.

“Theoretical loading capacity” and “loading capacity” as used herein refer to the amount of photosensitizers loaded per unit weight of the nanoparticle(s), with the former encompassing the calculated loading capacity and the former the empirical loading capacity. The nanoparticles described herein may have a loading capacity and/or theoretical loading capacity of up to 100% by weight, including 50 wt. %, 60 wt. %, 70 wt %, 80 wt. %, 90 wt. % and all integers included within these ranges.

The term “nanoparticle carrier” as used herein refers to small transport agents that can be modified in terms of size, charge, and shape to carry therapeutic agents to specific tissues.

The term “lipid nanoparticle” as used herein refers to organic-based nanoparticles that are solid colloidal particle systems in which drug material is either entrapped, encapsulated and dissolved in a lipidic matrix or adsorbed onto matrix.

The term “conjugated” as used herein refers to two or more molecules being linked together.

The term “liposome” as used herein refers to a small spherical vesicle having at least one lipid bilayer.

The term “monodispersed” as used herein refers to particles of uniform size.

The term “polydisperse” as used herein refers to particles with varying sizes.

The term “photoactive” as used herein refers to a molecule or chemical substance capable of a chemical or physical change in response to illumination.

The term “cytotoxic” as used herein means toxic to living cells.

The term “healthy cells” as used herein refers to cells that exhibit normal physiological function, morphology and viability, without signs of disease, malignancy, infection, genetic mutation or other pathological alterations. These cells present regulated proliferation, intact metabolic activity, genomic stability and appropriate responses to physiological signals.

The term “cancer cells” as used herein refer to abnormal cells that divide and grow uncontrollably.

The term “surfactant” as used herein refers to a substance that is added to a liquid to reduce the surface tension and increase the spreading and wetting properties.

The term “encapsulated” as used herein refers to the dBPD that is enclosed withing a surrounding lipid matrix/shell. dBPD is entrapped within a lipidic structure having a defined core-shell architecture yielding dBPD-NPs or FA-dBPD-NPs when part of the lipids used in the synthesis contain folic acid (FA).

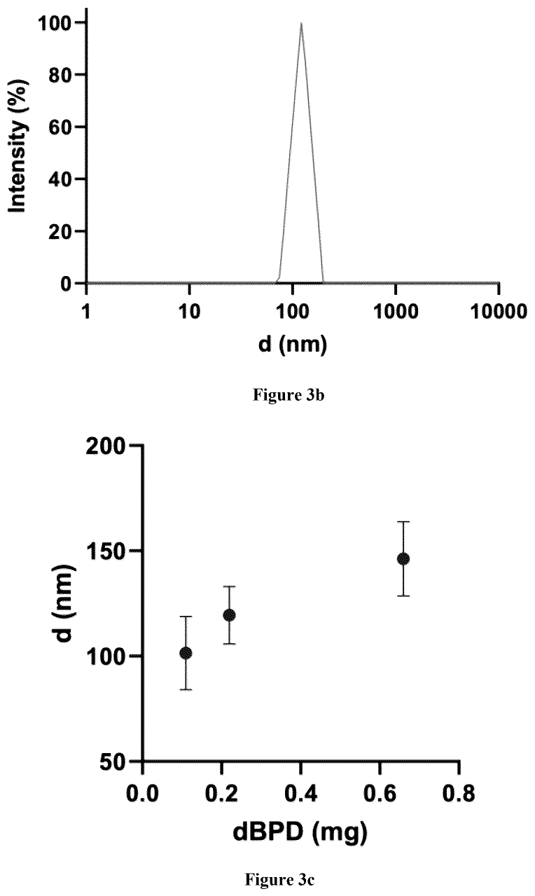

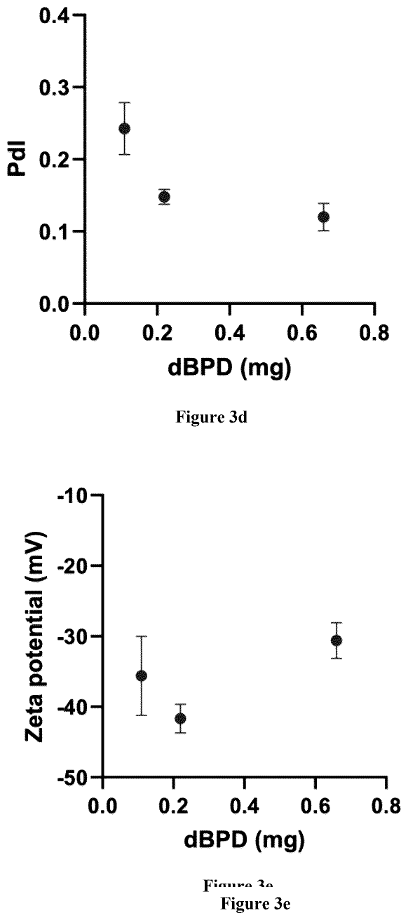

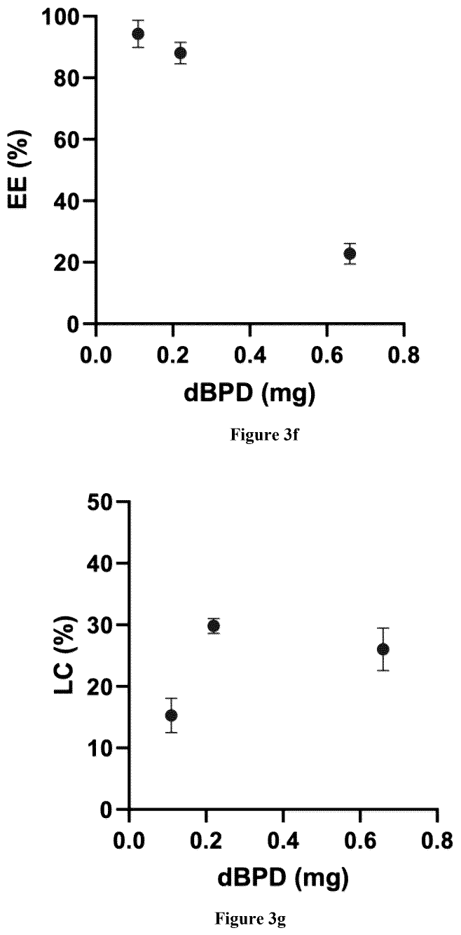

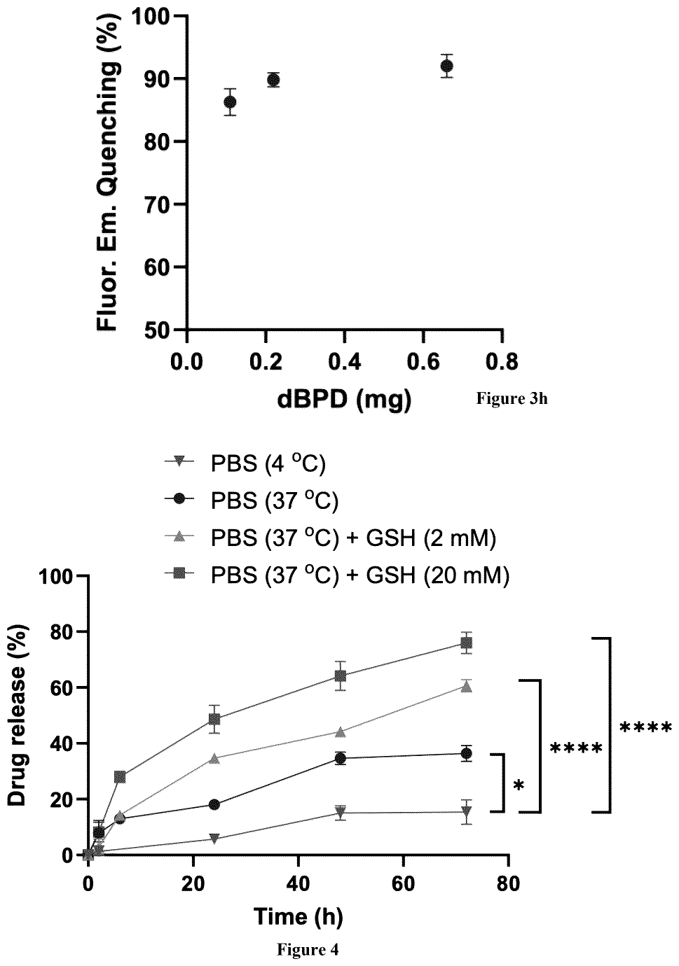

In some embodiments, “dBPD-loaded nanoparticle” is in the form as illustrated in FIG. 3A.

The term “folate receptors” as used herein refers to proteins on the cell surface that bind to folate that bind to folic acid.

The term “selectively cytotoxic to cancer cells only” as used herein refers to the property of FA-dBPD-NPs to induce cell death (cytotoxicity) in cancerous or malignant cells, while exhibiting minimal or no cytotoxic effects on non-cancerous, healthy, or normal cells under comparable conditions. In this case, the selectivity is achieved towards folate receptor-overexpressing cells via the use of folic acid. Cancer cells that exhibit significantly higher levels of folate receptor expression on their surface relative to normal, non-cancerous cells of the same tissue type. This overexpression enhances the cellular uptake of folic acid and its derivatives (FA-dBPD-NPs). In contrast, the cancer cells or other cells without overexpression of folate receptors present a limited folate-mediated uptake and reduces the accumulation of FA-dBPD-NPs. This difference is utilized to achieve selective targeting of cancer cells overexpressing folate receptors through folate-conjugated agents/delivery systems.

The term “endoplasmic reticulum” as used herein refers to a network of membranous tubules within the cytoplasm of a eukaryotic cell, continuous with the nuclear membrane.

The abbreviation “BPD” means benzoporphyrin derivative.

The abbreviation “FDA” means United States Food and Drug Administration.

The abbreviation “dBPD” means dimeric benzoporphyrin derivative.

The abbreviation “PEG” means polyethylene glycol.

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Moreover, all ranges disclosed herein are to be understood to encompass any and all subranges (including all fractional and whole values) subsumed therein.

### LIST OF EMBODIMENTS

1. A dimeric benzoporphyrin derivative (dBPD) of Formula I:

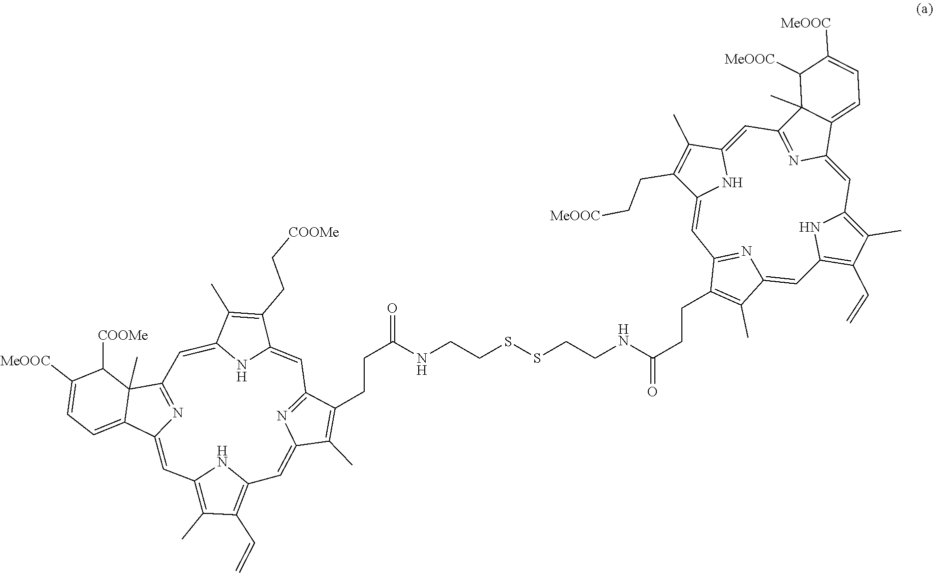 [SMILES: C=Cc1c(C)c2cc3nc(cc4[nH]c(cc5nc(cc1[nH]2)C(C)=C5CCC(=O)NCCSSCCNC(=O)CCC1=C(C)c2cc5[nH]c(cc6nc(cc7[nH]c(cc1n2)c(CCC(=O)OC)c7C)C1(C)C6=CC=C(C(=O)OC)C1C(=O)OC)c(C)c5C=C)c(CCC(=O)OC)c4C)C1(C)C3=CC=C(C(=O)OC)C1C(=O)OC]

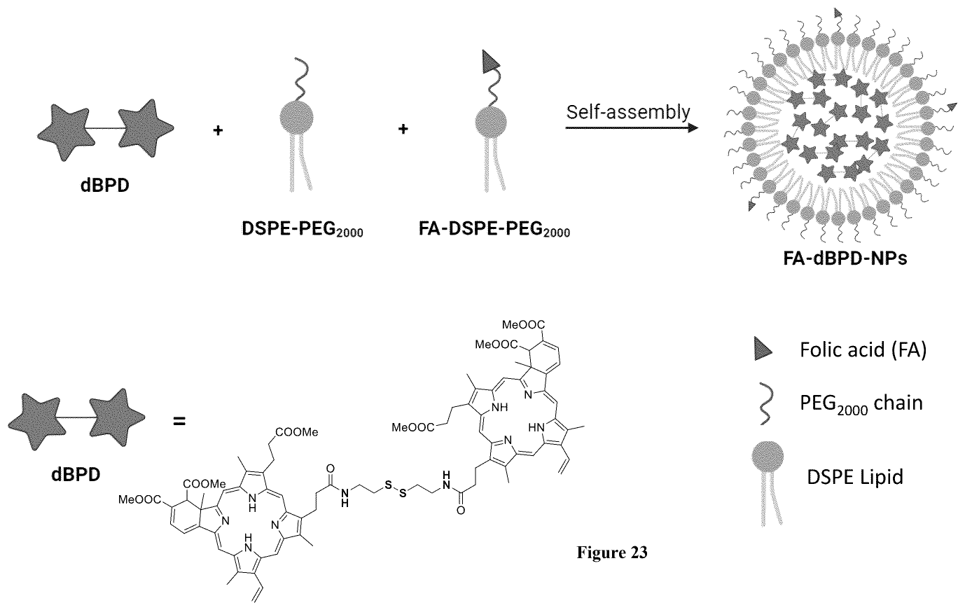

2. The dimeric molecule of embodiment 1, wherein said dBPD is formed by conjugating two benzoporphyrin derivative molecules using cystamine as the linker moiety by EDC/HOBt coupling reaction between the amine groups in the cystamine and the carboxyl group on BPD. The disulfide bond was specifically selected due to its potential to induce release upon reaction with GSH.
3. The dimeric molecule of embodiment 1, wherein said dBPD is highly hydrophobic (fluorescence emission of dBPD was fully quenched in the presence of 60% H₂O whereas BPD required 80% H₂O to reach the total quenching of the fluorescence signal).
4. A nanoparticle composition, said nanoparticle composition comprising a dBPD of any of the preceding embodiments and polyethylene glycol (PEG): DSPE-mPEG2000, DSPE-mPEG1000, DSPE-mPEG750, DSPE-mPEG5000, Cholesterol-mPEG2000, mPEG-DSG, mPEG-DMG, mPEG-PLGA, mPEG-PLA, among others, wherein said nanoparticle composition is in the form of a dBPD-loaded nanoparticle: a lipid-based self-assembled nanomedicine formed by a dimeric form of BPD. In some embodiments, said nanoparticle composition is in the form of a dBPD-loaded nanoparticle as illustrated in FIG. 3a.
5. The nanoparticle composition of embodiment 4, wherein said PEG is arranged in the form of a liposome. In some embodiments, PEG is arranged in the form of a liposome as illustrated in FIG. 3a and FIG. 23.
6. The nanoparticle composition of embodiment 4, wherein said PEG has a molecular weight of about 750 Da to about 5,000 Da
7. The nanoparticle composition of embodiment 4, wherein said dBPD is encapsulated within said nanoparticle. In some embodiments, dBPD is encapsulated within said nanoparticle as illustrated in FIG. 3a and FIG. 23.
8. The nanoparticle composition of embodiment 7, wherein said dBPD is encapsulated within said nanoparticle in the amount of about 0.1 mg to about 0.7 mg. With a preferred amount of 0.22 mg of dBPD in the synthesis.
9. The nanoparticle composition of any of the preceding embodiments, wherein said nanoparticle composition is monodispersed.
10. The nanoparticle composition of any of the preceding embodiments, wherein said nanoparticle is a spherical shape with a size of about 135 nm to about 141 nm.
11. The nanoparticle composition of any of the preceding embodiments, wherein said nanoparticle composition further comprises folic acid.
12. The nanoparticle composition of embodiment 11, wherein said folic acid is tethered to said PEG on the outer portion of said capsule. In some embodiments, folic acid is tethered to said PEG on the outer portion of said capsule as illustrated in FIG. 23.
13. The nanoparticle composition of embodiment 11, wherein said folic acid is present in the amount of about 0 wt % to about 10 wt %.
14. The nanoparticle composition of embodiment 12, wherein said folic acid is tethered to said PEG and is present on at least 50% or more of the surface of said nanoparticle.
15. The nanoparticle composition of embodiment 12, wherein said folic acid is tethered to said PEG and is present on at least 60% or more of the surface of said nanoparticle.
16. The nanoparticle composition of embodiment 12, wherein said folic acid is tethered to said PEG and is present on at least 70% or more of the surface of said nanoparticle.
17. The nanoparticle composition of embodiment 12, wherein said folic acid is tethered to said PEG and is present on at least 80% or more of the surface of said nanoparticle.
18. The nanoparticle composition of embodiment 12, wherein said folic acid is tethered to said PEG and is present on at least 90% or more of the surface of said nanoparticle.
19. The nanoparticle composition of embodiment 12, wherein said folic acid is tethered to said PEG and is present on at least 99% or more of the surface of said nanoparticle.
20. The nanoparticle composition of any of the preceding embodiments, wherein said nanoparticle is photoactive.
21. The nanoparticle composition of embodiment 20, wherein said nanoparticle is photoactivated at about 435 nm or about 690 nm to form a photoactivated nanoparticle.
22. The nanoparticle composition of embodiment 20, wherein said photoactivated nanoparticle is cytotoxic.
23. The nanoparticle composition of embodiment 22, wherein said photoactivated nanoparticle is selectively cytotoxic to cancer cells only.
24. The nanoparticle composition of embodiment 22, wherein said photoactivated nanoparticle is selectively not cytotoxic to healthy cells.
25. A formulation, said formulation comprising a nanoparticle composition of any of the preceding embodiment and one or more pharmaceutically acceptable excipients.
26. The formulation of embodiment 25, wherein said pharmaceutically acceptable excipient is water.
27. The formulation of any of the preceding embodiments wherein said formulation is administered parenterally or intravenously to a patient or a subject in need thereof.
28. The formulation of any of the preceding embodiments, wherein said formulation is photoactive.
29. The formulation of embodiment 27, wherein said formulation is cytotoxic when said formulation is photoactivated.
30. The formulation of embodiment 29, wherein said formulation is photoactivated at about 690 nm.
31. The formulation of embodiment 29, wherein said photoactivated formulation is selectively cytotoxic to cancer cells only.
32. The formulation of embodiment 30, wherein said photoactivated formulation is selectively delivered to the endoplasmic reticulum of said cancer cells.
33. The formulation of embodiment 31, wherein said photoactivated formulation is selectively not cytotoxic to healthy cells.
34. A method of making a dimeric molecule, said method comprising linking two benzoporphyrin derivative (BPD) moieties. A stock solution of cystamine was prepared by dissolving it (6.4 mg, 42.03 μmol) in dimethylformamide (DMF) (8.5 mL). N,N-Diisopropylethylamine (DIPEA) (2 mL, 11.48 mmol) was added to the previous mixture and stirred for 15 min. Next, the previous stock solution (4.5 mL, 18.01 μmol of cystamine) was added to a vial containing 1-Ethyl-3-(3-dimethylaminopropyl) carbodiimide (EDC) (60 mg, 312.99 μmol), 1-Hydroxybenzotriazole (HOBt) (29.1 mg, 215.36 μmol) and benzoporphyrin derivative (BPD, US Pharmacopeia) (26.5 mg, 36.87 μmol) in DMF (1.5 mL). The reaction mixture was left stirring at 35° C. for 48 h. After this time, the product was extracted using diethyl ether/H₂O mixture and the organic phase was washed 3 times with H2O to remove the excess DMF and the final product was dried under vacuum. To further purify the dBPD, the product was dissolved in dichloromethane (DCM) (2 mL) and loaded into a preparative TLC plate (TLC Silica Gel 60 F254 Multiformat, Millipore Sigma) run using ethyl acetate. The product was extracted from the TLC and separated from the silica by extraction with diethyl ether, followed by 5 washes centrifugating at 1000 rpm for 5 min. After evaporation of the solvent, dBPD was obtained as a dark green powder (14.6 mg, 51%). 1H (600 MHZ, DMSO-d6, δ): 9.90 (1H, s), 9.83-9.81 (1H, m), 9.73-9.61 (5H, m), 9.33-9.28 (2H, m), 8.43-8.38 (1H, m), 8.32-8.16 (1H, m), 8.06-8.02 (1H, m), 7.81-7.77 (5H, m), 6.50-6.04 (5H, m), 5.27-5.25 (2H, m), 4.30-4.06 (8H, m), 3.93-3.91 (10H, m), 3.63 (2H, s), 3.58 (4H, s), 3.54-3.44 (15H, m), 2.96-2.93 (9H, m), 2.90 (10H, s), 2.74 (4H, s) (Figure S1). MS-MALDI was obtained in linear/positive mode using DHB as the matrix in a Bruker MALDI-TOF (Figure S2). HRMS (ESI) m/z: [M+H]⁺ calcd for C86H₉₂N10014S2 1553.6309. found, 1553.5861. The presence of different isomers was demonstrated by liquid chromatography-electrospray ionization mass spectrometry (LC-ESI+) as shown in Figure S3. The absorption and the fluorescence emission spectra of the dBPD were recorded (Cytation 5, BioTek) in dimethyl sulfoxide (DMSO) and with different amounts of H2O (0, 20, 40, 60, 80 and 98%).
35. The method of embodiment 34, wherein two BPD moieties are linked using cystamine to obtain a compound of Formula I.
36. The method of any of the preceding embodiments wherein the carboxyl group on said BPD is coupled to an amine group on said cystamine.
37. A method of making a dBPD-loaded nanoparticle, said method comprising:
   a. dissolving dBPD in an organic polar solvent;
   b. mixing solution of step a with a pegylated lipid;
   c. evaporating said organic solvent from the mixture of step b to form a solid;
   d. hydrating the solid with an aqueous solution; and
   e. freeze-thawing the solution of step d to form dBPD-NPs.
38. The method of embodiment 37, wherein said freeze-thawing comprised treating said solution with temperatures alternating between 4° C. and 45° C.
39. The method of embodiment 37, wherein said solvent is chosen from chloroform, methanol, ethanol, acetone, dichloromethane, tetrahydrofuran, etc.
40. The method of embodiment 37, wherein said pegylated lipid is distearoylphosphatidylethanolamine-methoxy polyethylene glycol.
41. A method of making a folic acid functionalized dBPD-loaded nanoparticle, said method comprising:
   a. dissolving dBPD in an organic solvent;
   b. mixing solution of step a with a pegylated lipid;
   c. adding folic acid to the mixture of step b;
   d. evaporating said organic solvent from the mixture of step c to form a solid;
   e. hydrating the solid with an aqueous solution; and
   f. freeze-thawing the solution of step e to form folic acid functionalized dBPD-NPs.
42. The method of embodiment 41, wherein said freeze-thawing comprised treating said solution with temperatures alternating between 4° C. and 45° C.
43. The method of embodiment 41, wherein said solvent is chosen from chloroform, methanol, ethanol, acetone, dichloromethane, tetrahydrofuran, etc.
44. The method of embodiment 41, wherein said pegylated lipid is distearoylphosphatidylethanolamine-methoxy polyethylene glycol.
45. A method of treating cancer, said method comprising administering a pharmaceutically effective amount of dBPD or a formulation of any of the preceding claims to a patient or subject in need thereof.
46. The method of embodiment 45, wherein said cancer involves overexpression of folate receptors.
47. The method of embodiment 45, wherein said dBPD or said formulation of any of the preceding claims is parenterally administered.
48. The method of embodiment 47, wherein said dBPD or formulation of any of the preceding claims is administered intravenously, orally, topically, intraperitoneally, intraarterially or intratumorally.
49. The method of embodiment 45, wherein said formulation releases dBPD from the dBPD-loaded nanoparticle in the presence of glutathione (GSH)
50. The method of embodiment 49, wherein said GSH is present in the amount of about 2 mM to about 20 mM.
51. The method of embodiment 45, wherein said formulation of any of the preceding claims is photoactive.
52. The method of embodiment 45, wherein said formulation of any of the preceding claims is cytotoxic when said formulation is photoactivated.
53. The method of any of embodiment 52, wherein said formulation of any of the preceding claims is photoactivated at about 435 nm to about 690 nm.
54. The method of embodiment 52, wherein said photoactivated formulation is selectively cytotoxic to cancer cells only.
55. The method of embodiment 52, wherein said photoactivated formulation is selectively delivered to the endoplasmic reticulum of said cancer cells.
56. The method of embodiment 45, wherein said cancer comprises cancer cells that overexpress folate receptors.
57. The method of embodiment 56, wherein said cancer is lung cancer, ovarian cancer, endometrial cancer, or breast cancer.
58. The method of embodiment 57, wherein said cancer is ovarian cancer.

### EXAMPLES

The following examples are provided solely to illustrate the present invention and are not intended to limit the scope of the invention, described herein.

### Example 1. Synthesis and Characterization of the Dimeric Form of BPD

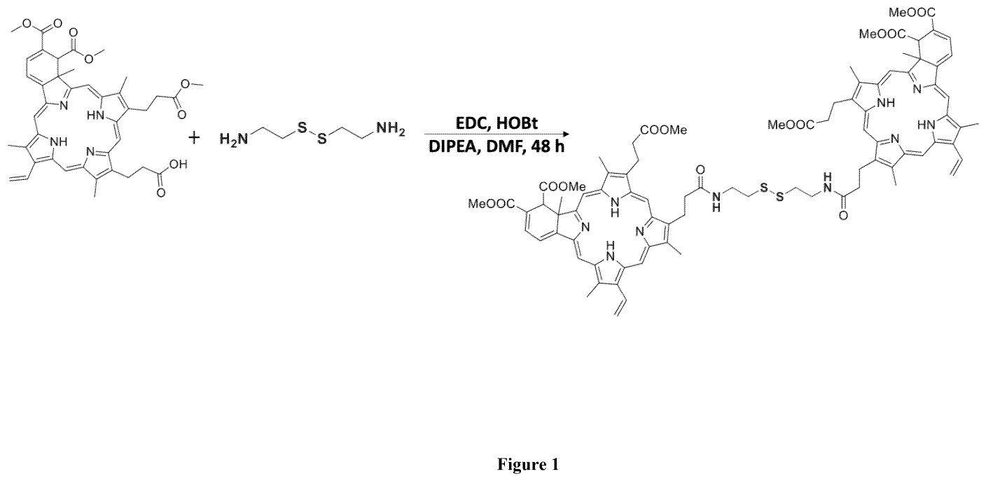

A dimeric form of benzoporphyrin derivative (dBPD) was synthesized using cystamine as the linker moiety by EDC/HOBt coupling reaction between the amine groups in the cystamine and the carboxyl group on BPD (FIG. 1). The disulfide bond was specifically selected due to its potential to induce release upon reaction with GSH.

A stock solution of cystamine was prepared by dissolving 6.4 mg (42.03 μmol) in DMF (8.5 mL). DIPEA (2 mL, 11.48 mmol) was added to the previous mixture and stirred for 15 min. Next, 4.5 mL of the previous stock solution (18.01 μmol of cystamine) was added to a vial containing EDC (60 mg, 312.99 μmol), HOBt (29.1 mg, 215.36 μmol) and BPD (26.5 mg, 36.87 μmol) in DMF (1.5 mL). The reaction mixture was left stirring at 35° C. for 48 h. After this time, the product was extracted using diethyl ether/H₂O mixture and the organic phase was washed 3 times with H₂O to remove the excess DMF and the final product was dried under vacuum. To further purify the dBPD, the product was dissolved in 2 mL DCM and loaded into a preparative TLC plate (TLC Silica Gel 60 F254 Multiformat, Millipore Sigma) run using ethyl acetate. The product was extracted from the TLC and separated from the silica by extraction with diethyl ether, followed by 5 washes centrifugating at 1000 rpm for 5 min. After evaporation of the solvent, dBPD was obtained as a dark green powder (14.6 mg, 51%).

MS-MALDI was obtained in linear/positive mode using DHB as the matrix in a Bruker MALDI-TOF. HRMS (ESI+) calc. for dBPD [M+H]+: 430.1879; found: 430.1807.

The absorption and the fluorescence emission spectra of the dBPD were recorded (Cytation 5, BioTek) in DMSO and with different amounts of H₂O (0, 20, 40, 60, 80 and 98%).

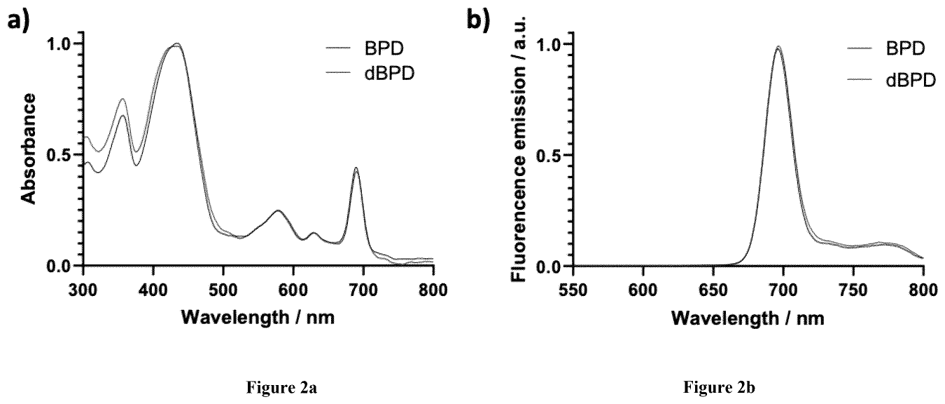

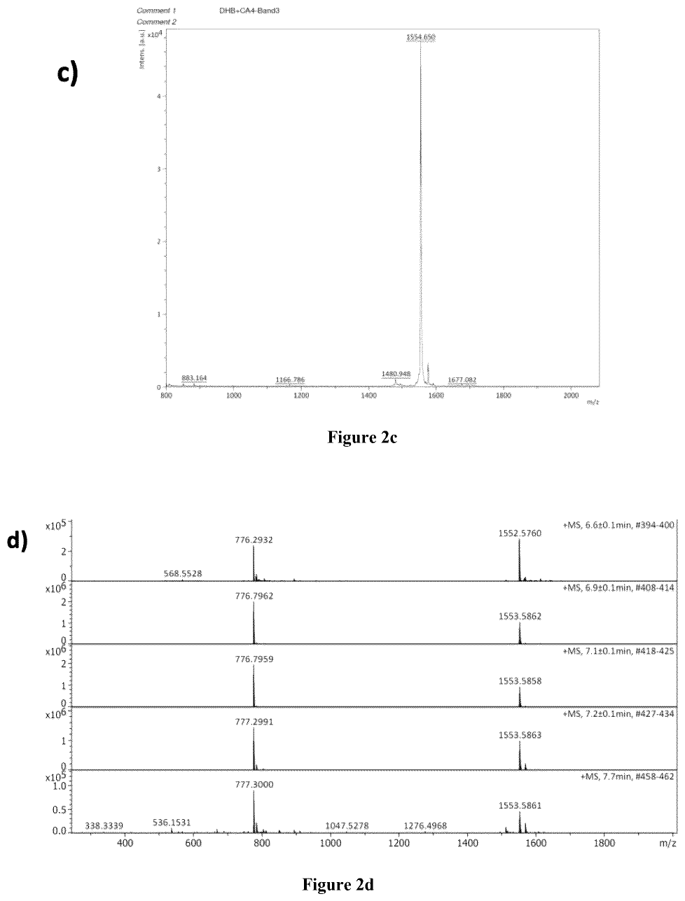

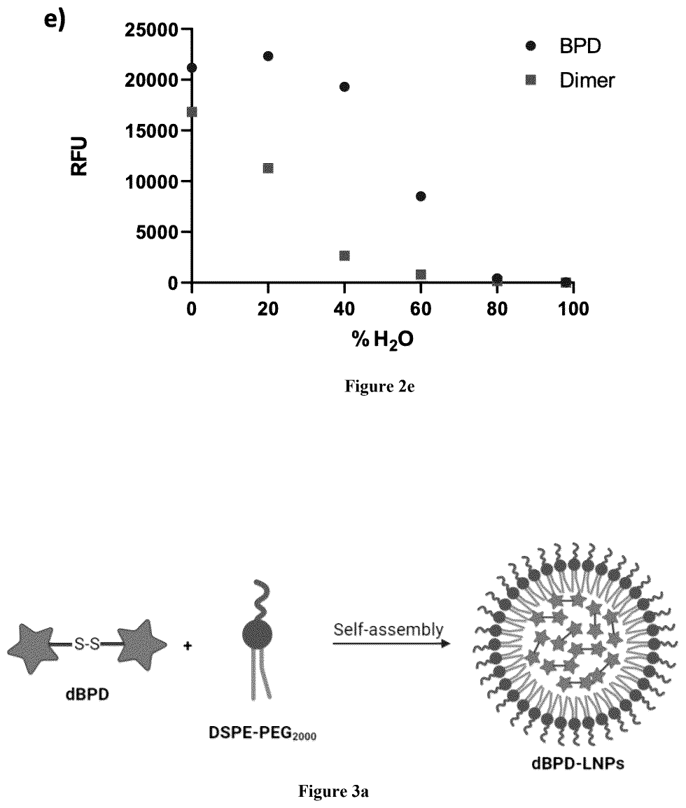

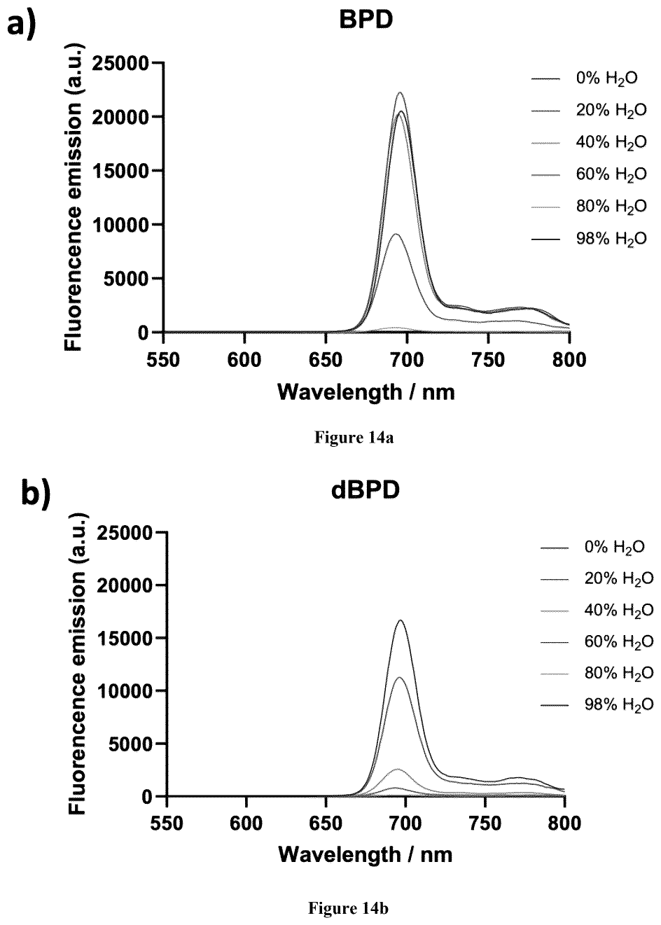

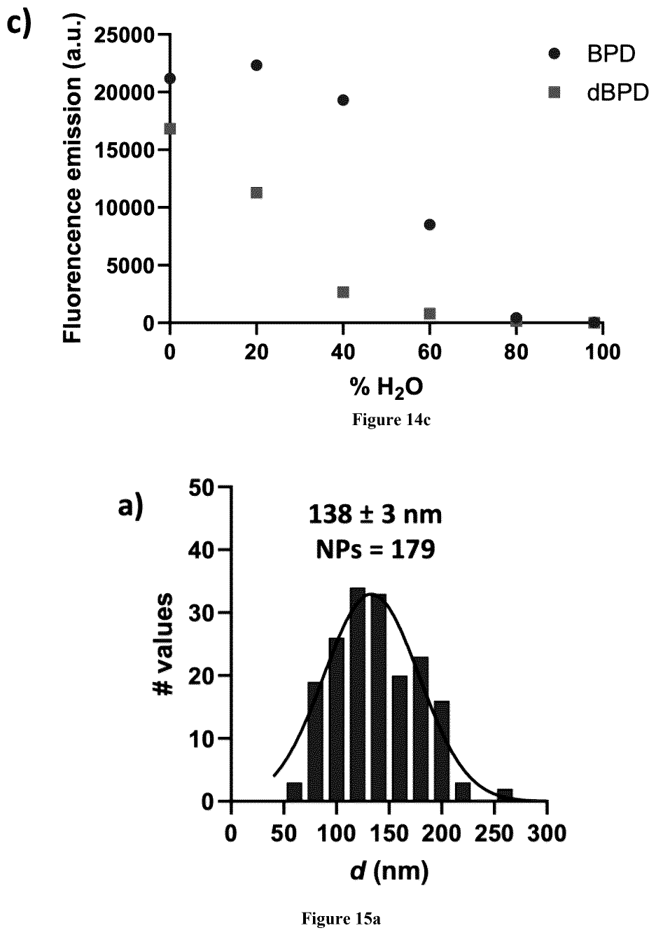

The synthesis of the dimeric form of BPD was confirmed through analysis using mass spectrometry (MS), UV-Vis spectroscopy and fluorescence emission (FIG. 2). The compound formed was also characterized by liquid chromatography-ESI+ showing five different peaks at the same molecular weight indicating structural differences (FIG. 2d). This is likely due to the presence of different stereoisomers of the dBPD, at least five. The absorption and the fluorescence emission spectra of dBPD (red, FIGS. 2a and 2b) showed the same characteristic peaks of BPD (blue, FIGS. 2a and 2b) with no shift after conjugation. To investigate the differences in hydrophobicity between BPD and dBPD, the fluorescence emission spectra of both drugs were recorded at different DMSO/H₂O ratios (FIG. 2e and FIG. 14) showing that the fluorescence emission of dBPD was fully quenched in the presence of 60% H₂O whereas BPD required 80% H₂O to rich the total quenching of the fluorescence signal. These results prove the superior hydrophobicity of the dBPD for the most efficient encapsulation in organic nanoparticles (NPs) like liposomes, micelles or polymeric NPs.

### Example 2. Synthesis and Characterization of dBPD-Loaded Nanoparticles (dBPD-NPs)

dBPD-NPs, 1% FA-dBPD-NPs and 10% FA-dBPD-NPs were formed following the freeze-thaw method. Briefly, distearoylphosphatidylethanolamine-methoxy polyethylene glycol (DSPE-mPEG2000; Avanti Polar Lipids) and dBPD diluted in chloroform were mixed at different molar ratios of DSPE-mPEG200:dBPD to find the optimal nanoassembly. Nanoparticles with molar ratios of 1:0.5 (0.11 mg dBPD), 1:1 (0.22 mg dBPD) and 1:3 (0.66 mg dBPD) were synthesized and characterized. 1,2-Distearoyl-sn-glycero-3-phosphoethanolamine-N-[folate (polyethylene glycol)] (FA-DSPE-mPEG2000; Nanosoft Polymers) at 1 or 10% molar was added to a molar ratio of DSPE-mPEG2000:dBPD of 1:1 to yield 1% FA-dBPD-NPs and 10% FA-dBPD-NPs, respectively. Chloroform from all lipid mixtures was removed by rotary evaporation to create a thin film. The resulting dried films were hydrated with ultrapure deionized water (Invitrogen) before freeze-thaw cycling alternating between 4° C. and 45° C. After this, the NPs were filtered through a 0.22 μm sterile filter unit (Millex®-MP Filter Unit, Millipore Sigma) and dialysed against 1× phosphate-buffered saline (PBS) at 4° C. to remove unencapsulated agents (Spectra/Por, MWCO 300 kDa, Spectrum Laboratories Inc.). Samples were stored at 4° C. until use. The dBPD concentration was determined using ultraviolet-visible (UV-Vis) spectroscopy using calculated molar extinction coefficients of 82,416 cm⁻¹ M⁻¹ and 36,462 cm⁻¹ M⁻¹ at 435 and 690 nm, respectively. NanoBrook Omni (Brookhaven Instruments) was used to determine particle size and polydispersity index. Zeta View (Particle Metrix ZetaView Mono) was used to determine the nanoparticle concentration of the samples and zeta potential. The loading capacity (LC) was calculated as the percentage of the amount of dBPD loaded (mg) per unit weight of the nanoparticle (mg dBPD+mg DSPE-PEG). The encapsulation efficiency (EE) was calculated as the percentage of the amount of dBPD loaded by the amount of dBPD encapsulated in the NPs. The fluorescence emission spectra were recorded (λ_{exc}=435 nm) in PBS and DMSO diluting 5 μL of the sample in 200 μL of the solvent. The quenching was calculated as the percentage of the difference in fluorescence emission intensity of dBPD at 690 nm in DMSO and PBS by the fluorescence emission intensity in DMSO. The size and morphology of these NPs were determined by Cryogenic Transmission Electron Microscopy (Cryo-TEM, JEOL JEM-2100 LaB6, 100 kV). The cryo-TEM samples were prepared by adding 5 μL of the sample onto a Lacey carbon grid (Ted Pella) previously treated and vitrified for 10 s in a semi-automated cryogenic plunge freezer. The colloidal stability of the NPs was investigated for 6 months at storage conditions (dark, 4° C.) by measuring the particle size and polydispersity index.

The dBPD-loaded nanoparticles (dBPD-NPs) were prepared using the thin-film hydration and freeze-thaw method with a consequent filtration and dialysis for purification. The dBPD was self-assembled in NPs and stabilized with the PEGylated lipids yielding dBPD-NPs (FIG. 3a). Distearoylphosphatidylethanolamine-methoxy polyethylene glycol (DSPE-mPEG2000, 67%, w/w) was used to form PEGylated NPs in the presence of the dimeric form of BPD. DSPE-mPEG2000 is a widely used phospholipid-polymer conjugate in drug delivery applications due to its high biocompatibility and biodegradability. Nanoparticles prepared with DSPE-mPEG2000 have shown prolonged blood circulation time, improved stability and enhanced encapsulation efficiency. PEGylated NPs could potentially reduce the clearance of the NPs by the reticuloendothelial system and thus improve the colloidal stability of the nanoparticles. The formation of NPs was demonstrated by dynamic light scattering (DLS) showing a unique peak centered at around 100 nm (FIG. 3b).

To optimize the synthesis of the dBPD-NPs, three different concentrations of dBPD (0.11, 0.22 and 0.66 mg) were synthesized, fixing the concentration of DSPE-PEG (0.44 mg). The DSPE-PEG concentration used in the synthesis of the dBPD-NPs was selected. The resulting dBPD-NPs were characterized using DLS, UV-Vis, fluorescence emission and Zetaview to measure the hydrodynamic diameter (d), polydispersity index (PdI), zeta potential, encapsulation efficiency (EE), loading capacity (LC) and fluorescence emission quenching. Results showed an increase in particle size (FIG. 3c) and a decrease in PdI (FIG. 3d) when higher amounts of dBPD (0.22 and 0.66 mg) were used in the synthesis, with the PdI remaining below 0.2. The high negative zeta potential (around-35 mV) of the different samples predicted high stability in circulation (FIG. 3e). The EE and the LC of dBPD were calculated by determining the concentration of dBPD using UV-Vis as described in the Supporting Information. Interestingly, the EE of the dBPD was above 80% for samples prepared using 0.11 and 0.22 mg and dropped to 20% when 0.66 mg of dBPD was added to the synthesis (FIG. 3f). The LC of the resulting dBPD-NPs was found to be 30% when 0.22 mg of dBPD was used in the synthesis being the highest among the three conditions (FIG. 3g). Another parameter to consider when encapsulating photosensitizers in nanoparticles is the fluorescence emission quenching produced by the proximity of the molecules and the presence of an aqueous solvent. The fluorescence emission intensity of dBPD-NPs in PBS at 690 nm was compared to that of the dBPD-NPs in DMSO. As expected, when a higher concentration of dBPD was used in the formation of the dBPD-NPs, a slightly enhanced quenching was observed (FIG. 3h). All these results suggest that the lipid film is effective in the encapsulation of the dBPD with a limit of encapsulation for higher amounts of the drug (0.66 mg) and high polydispersity for low concentrations of dBPD (0.11 mg). From these results, 0.22 mg of dBPD was chosen as the optimal amount of drug to be used in the preparation of the dBPD-NPs since it showed higher EE and LC as well as a PdI below 0.2. Cryogenic transmission electron microscopy (cryo-TEM) images of dBPD-NPs synthesized using 0.22 mg of dBPD showed a spherical shape with an average size of 138±3 nm (FIG. 15).

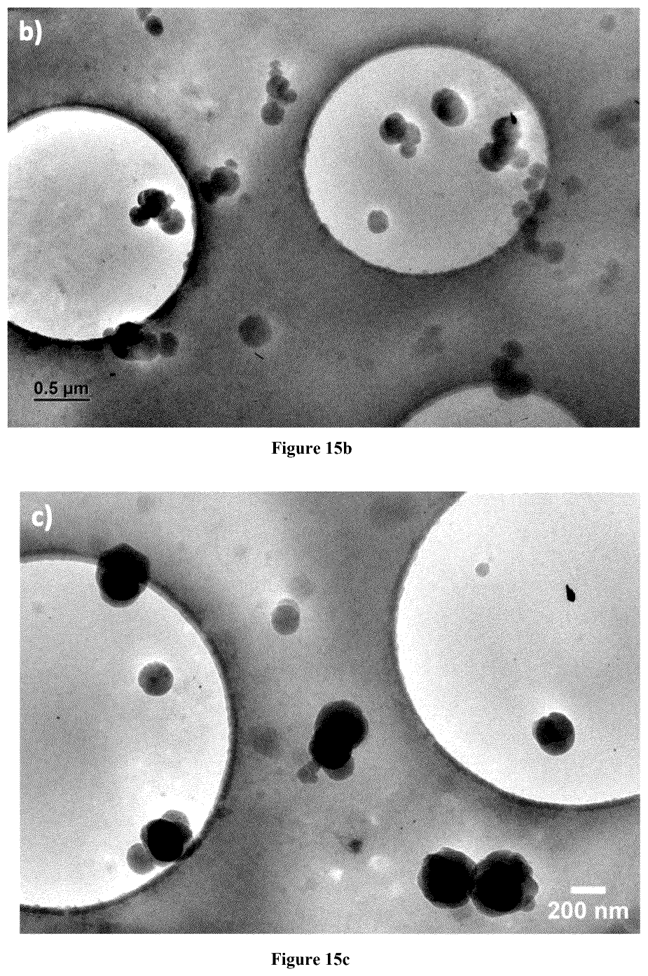

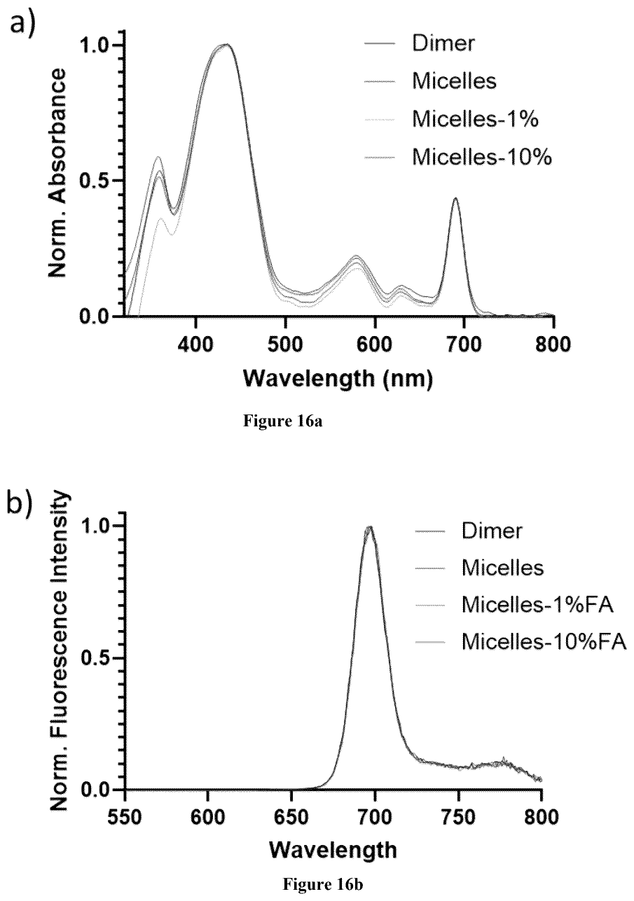

The absorbance and the fluorescence emission intensity spectra of dBPD-NPs showed the characteristic peaks of dBPD in both DMSO and PBS, demonstrating the successful encapsulation of the photosensitizer in the NPs (FIG. 16). The absorbance spectrum of dBPD-NPs showed a higher absorption signal compared to free dBPD at the same concentration of the drug. Additionally, the fluorescence emission spectrum of dBPD-NPs showed a 136-fold higher fluorescence intensity than that of dBPD at 690 nm when samples were dispersed in PBS. These results demonstrate an efficient encapsulation of dBPD in the dBPD-NPs and a reduced quenching of the photosensitizer upon encapsulation in the NPs.

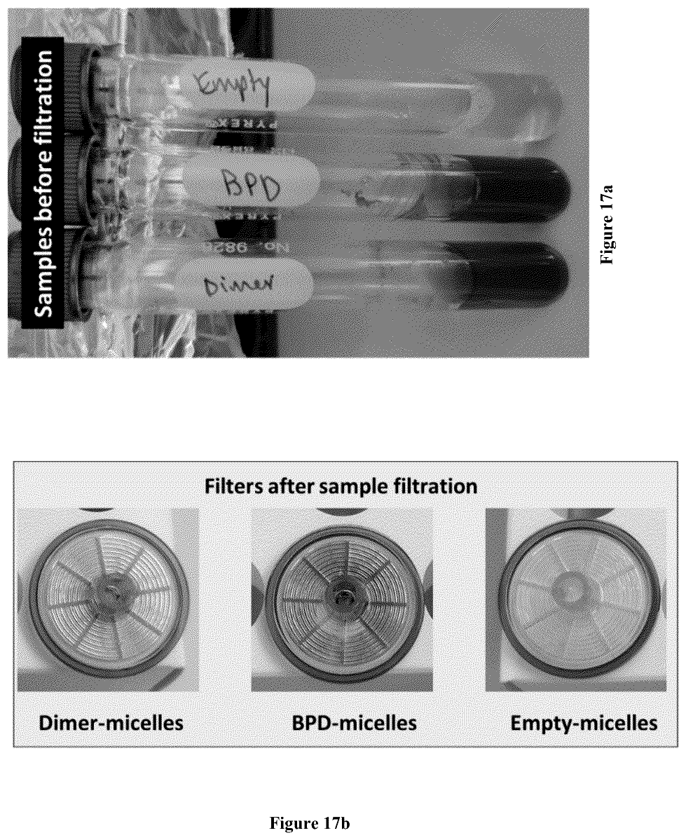

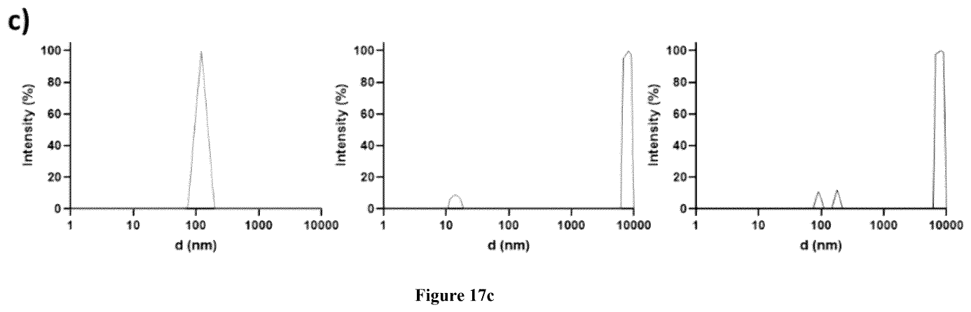

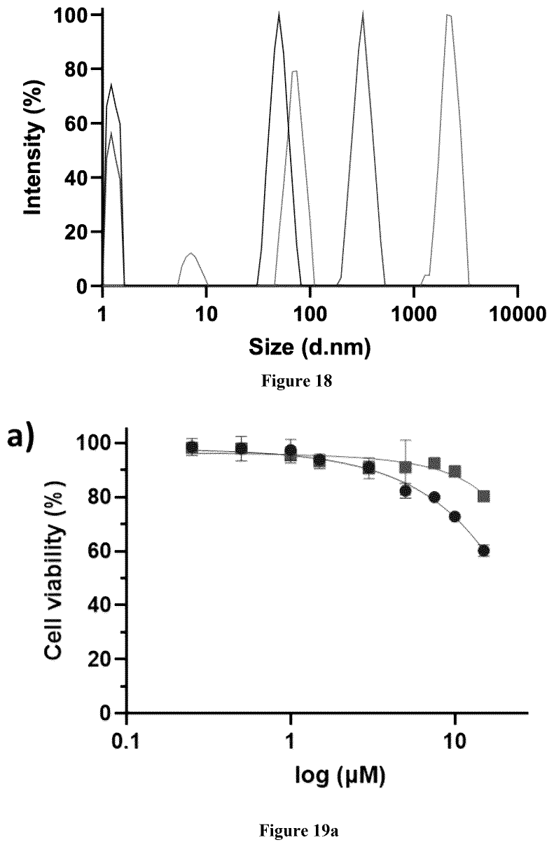

As discussed previously, the dBPD presents higher hydrophobicity compared to BPD as well as a neutral charge compared to the negative charge of the BPD, which makes dBPD more efficient for encapsulation into lipid-based NPs due to its self-aggregation effect driven by hydrophobic interactions. To compare the EE % of BPD versus dBPD, PEGylated NPs were synthesized using BPD at same concentration as dBPD following the previously described method. The results showed aggregates when the sample was filtered through a 0.22 μm sterile filter unit (FIG. 17b) and the filtrate showed aggregates when characterized by DLS (FIG. 17c). The synthesis of BPD-NPs was also attempted with a higher concentration of BPD (0.66 mg) and aggregates were also formed as confirmed by DLS (FIG. 18). Thus, the PEGylated NPs require a more hydrophobic drug such as the dBPD for its formation, efficient encapsulation and stability. Empty NPs were also synthesized by using only DSPE-mPEG2000 resulting in aggregates after the purification of the sample (FIG. 17c) showing that the concentration of PEGylated lipid is below the critical micellar concentration (CMC) and thus, not enough to form empty nanoparticles. These results demonstrate the advantage of using dBPD for the formation of these nanoparticles by self-assembly compared to BPD, as it improves drug loading efficiency and the stability of the final nanoformulation.

The final dispersion of dBPD-NPs presented an average concentration of 3.5×10¹¹±1.0×10¹¹ particles/mL and a concentration of dBPD of 69.8±6.4 μM. The molecules of BPD per nanoparticle in liposomes are estimated to be 485±75. For the dBPD-NPs developed in this work, it was calculated 218,602±20,718 BPD molecules per NPs which results in 450 times higher loading than for the liposomal formulation.

### Example 3. GSH-Triggered Drug Release Using dBPD-Loaded Lipid Nanoparticles (dBPD-NPs)

The backbone of the hydrophobic dBPD molecule consists of a disulfide bond which is able to trigger the drug release upon reaction with glutathione (GSH). Thus, GSH was used as an external redox stimulus to trigger the thiol-disulfide exchange reaction resulting in the disintegration of the nanoparticles and the release of the entrapped drug. To demonstrate the release of the drug in the presence of GSH, samples containing dBPD-NPs (45 μM) without and with 2 or 20 mM GSH were prepared. The samples were placed in a shaker at 37° C. and 50 rpm for 72 h. Sample aliquots were taken at different time points and the concentration was calculated using UV-Vis spectroscopy in DMSO. The results showed GSH concentration-dependent cumulative drug release profiles (FIG. 4). The absence of GSH in the storage conditions (4° C.) showed a minimum drug release potentially due to dilution of the sample in PBS. However, when the dBPD-NPs were stored at 37° C. and in the absence of GSH, a significant drug release was observed compared to storage conditions after 72 h but remained below 40%. In the presence of 2 mM GSH, the drug release after 72 h was shown to be 1.7-fold that in the absence of GSH and at 37° C. Upon increasing the GSH concentration to 20 mM, the release kinetics was accelerated showing a 2-fold enhancement compared to the sample without GSH at 37° C. These results demonstrate the GSH-triggered drug release in a concentration-dependent manner and could help to reduce the undesired exposure of the drug in the blood circulation and normal cells and, thus, reduce the dark toxicity of the photosensitizer in healthy tissues.

To study the GSH-induced dBPD release from the dBPD-NPs, the dBPD-NPs (45 μM, 1 mL) were placed in a dialysis tube (Spectra/Por, MWCO 300 kDa, Spectrum Laboratories Inc.) and submerged in 15 mL of PBS (pH 7.4) without or with GSH (2 mM or 20 mM). The tubes were then placed in a shaker (Incu-Shaker™ mini, Benchmark Scientific) at 37° C. and 50 rpm. Samples at storage conditions were also studied as controls in which the dBPD-NPs were placed at 4° C. The concentration of the sample aliquots (20 uL) was measured at different time points for 72 h using UV-Vis spectroscopy by diluting the aliquots in DMSO (180 uL). The volume of the sample remaining in the dialysis tube was recorded for each time point and used in the calculations of the drug released concentration.

### Example 4. Synthesis and Characterization of Folic Acid Engineered dBPD-NPs (FA-dBPD-NPs)

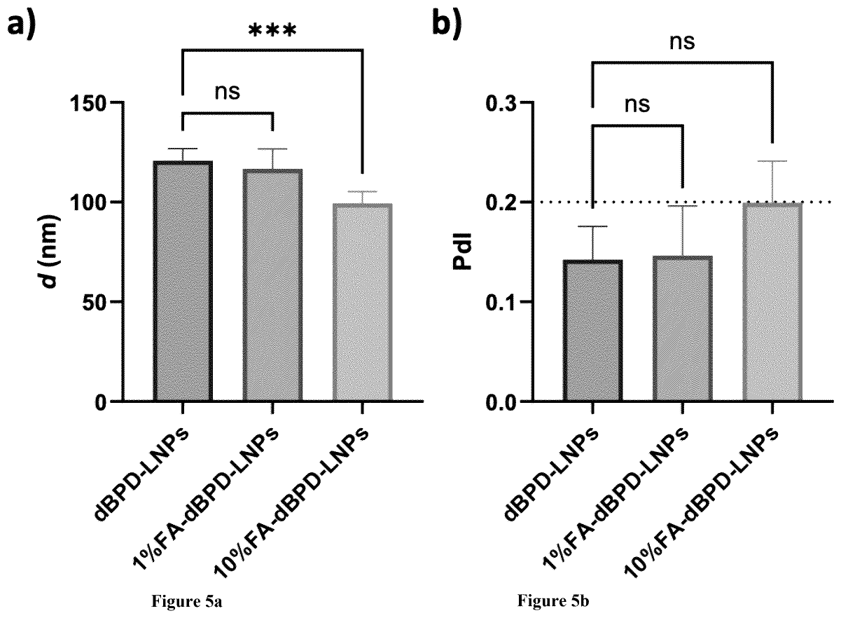

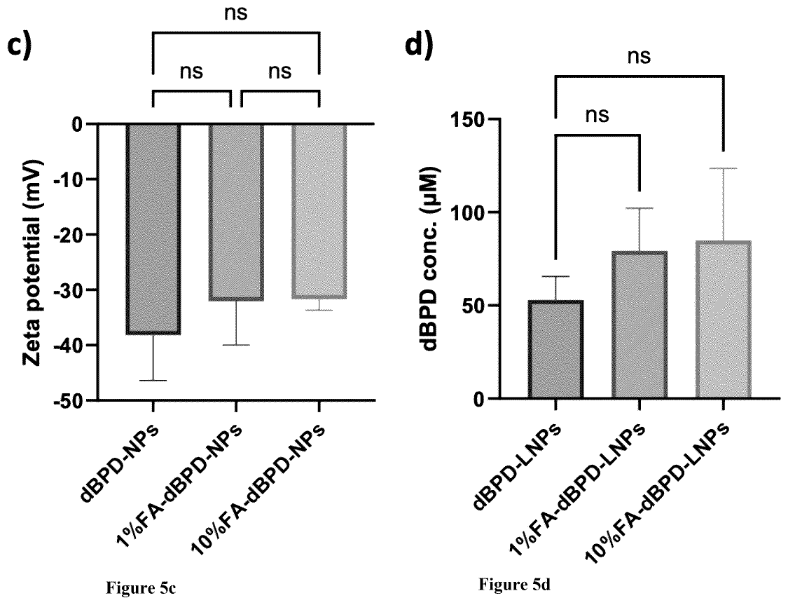

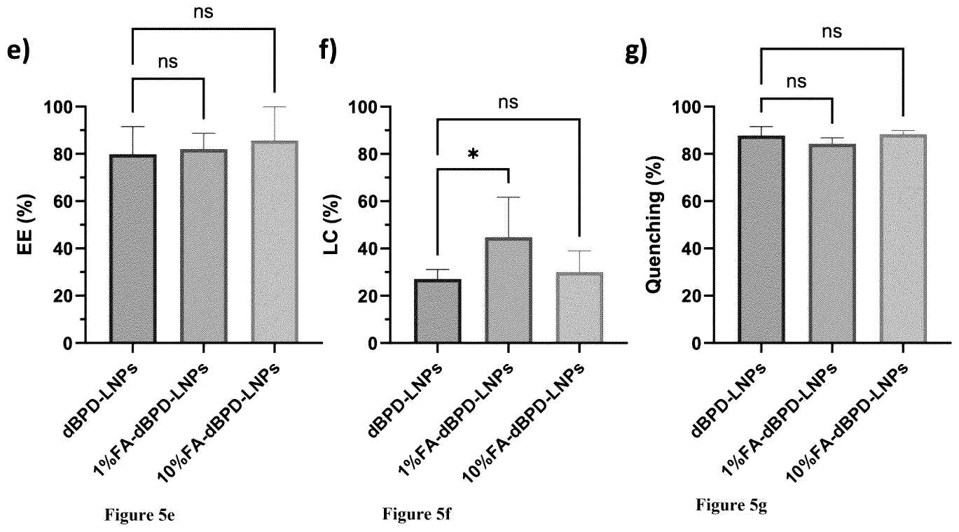

The folate receptor is overexpressed in most cancer cells, while its expression is limited in healthy cells. Targeted nanoparticles were synthesized by incorporating folic acid (FA) DSPE-PEG2000 lipids into the synthesis of the dBPD-NPs. To evaluate the optimal concentration of FA for the enhanced uptake in FR-overexpressed cells, dBPD-NPs with two concentrations of FA (1% and 10%) were prepared. Results showed the formation of NPs with both concentrations of FA with similar size and PdI as those without the targeting moiety (FIGS. 5a and 5b). The concentration of dBPD encapsulated in the NPs was higher with increasing concentrations of FA in the FA-dBPD-NPs (FIG. 5c). The EE values for all the nanoformulations showed similar results with no significant difference (FIG. 5d). Interestingly, 1% FA-dBPD-NPs showed a significantly higher LC value compared to dBPD-NPs (FIG. 5e). However, 10% FA-dBPD-NPs presented a similar LC value to that of dBPD-NPs. A fluorescence emission quenching of the dBPD above 80% was also observed in all the nanoparticles (FIG. 5f). These results demonstrate that both concentrations of FA tested, 1% and 10%, yielded stable and monodispersed dBPD-NPs.

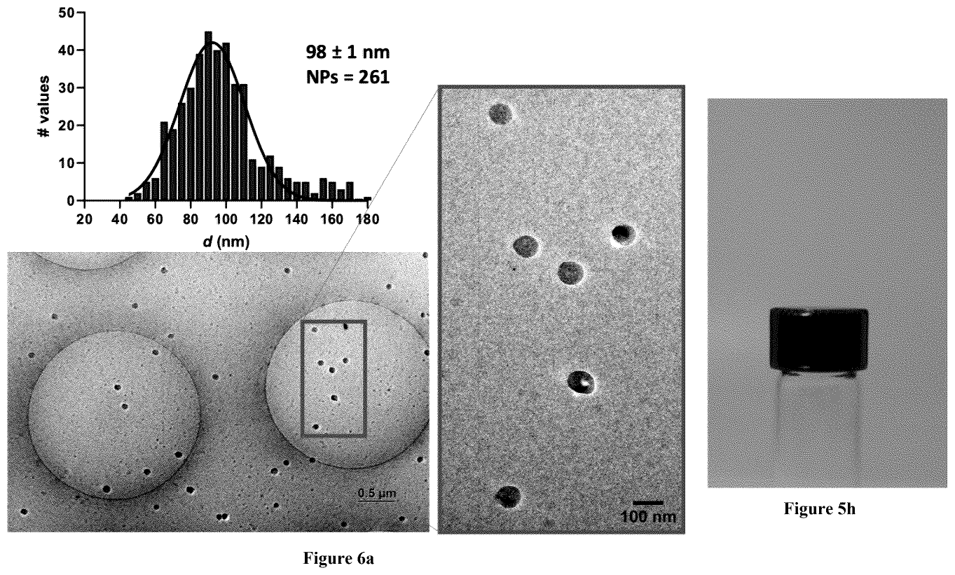

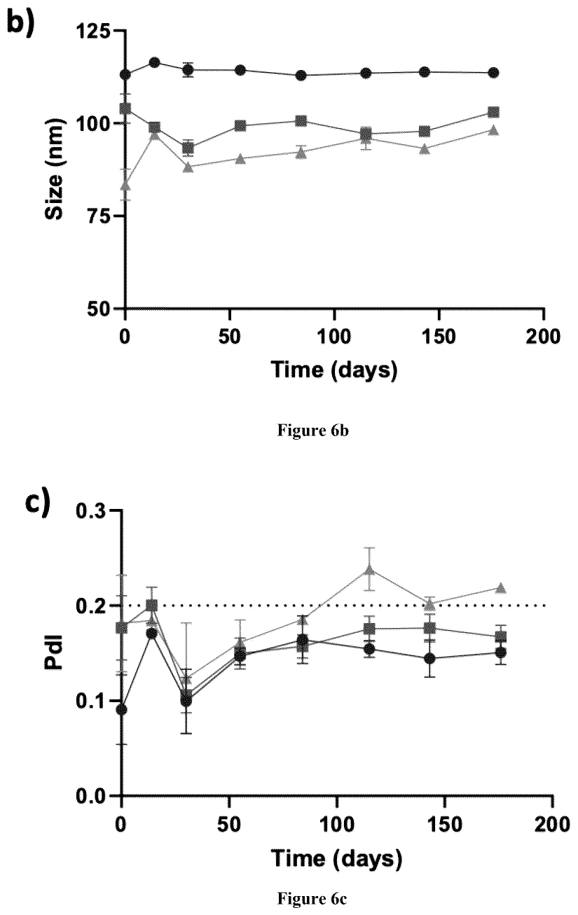

Cryogenic transmission electron microscopy (cryo-TEM) images of 1% FA-dBPD-NPs showed a spherical shape with an average size of 98±1 nm (FIG. 6a), which is very similar to the hydrodynamic diameter obtained by DLS (117±5 nm). Cryo-TEM images of the dBPD-NPs were also obtained showing similar results (FIG. 15). The colloidal stability of the nanoparticles was also studied during 6 months at storage conditions of dark and 4° C. in PBS. The diameter and PdI were recorded by DLS at different time points. Results showed very stable sizes for dBPD-NPs, 1% FA-dBPD-NPs and 10% FA-dBPD-NPs with maximum over time size variations of 3%, 10% and 16%, respectively (FIG. 6b). The PdI of these samples showed values below 0.2 for dBPD-NPs and 1% FA-dBPD-NPs and slightly above 0.2 for 10% FA-dBPD-NPs after 4 months (FIG. 6c). Overall, dBPD-NPs showed excellent stability with no aggregation and preservation of their size over 6 months at the storage conditions and the 1% FA-dBPD-NPs preserved that stability.

### Example 5. Photoactivity Efficiency and Singlet Oxygen Production of dBPD, dBPD-NPs, 1% FA-dBPD-NPs and 10% FA-dBPD-NPs

Singlet oxygen generation was determined using the Singlet Oxygen Sensor Green (SOSG) probe (Invitrogen). Human serum albumin 1% (HSA 1%) was prepared in PBS. Samples of dBPD, dBPD-NPs, 1% FA-dBPD-NPs, and 10% FA-dBPD-NPs were prepared in DMSO, PBS and HS 1% at 2 μM and were kept at 37° C. for 24 h. In a 96-well black bottom plate, 100 μL of the sample was mixed with 10 μL of SOSG stock (50 μM) and the fluorescence emission intensity was recorded for each sample and control (Δ_{exc}=504 nm/λₑₘ=525 nm). The samples were transferred to a 96-well clear bottom black wall plate. Light irradiation (690 nm, 40 J/cm², 150 mW/cm²) was performed using the ML8500 Modulight Laser System. The samples were transferred back to the 96-well black bottom plate and the fluorescence emission intensity was measured using the same parameters. Controls of solvent only (DMSO, PBS and HS 1%) and dark controls were prepared and measured for each sample.

Photoactivity is defined as the dBPD fluorescence emission maxima intensity of the sample in PBS divided by that in DMSO as previously described. Samples of dBPD, dBPD-NPs, 1% FA-dBPD-NPs, and 10% FA-dBPD-NPs were prepared in DMSO, PBS and HS 1% at 2 μM and were kept at 37° C. for 24 h. After this time, the absorption and the fluorescence emission (λ_{exc}=435 nm) spectra of dBPD were recorded for all the samples and the photoactivity was calculated.

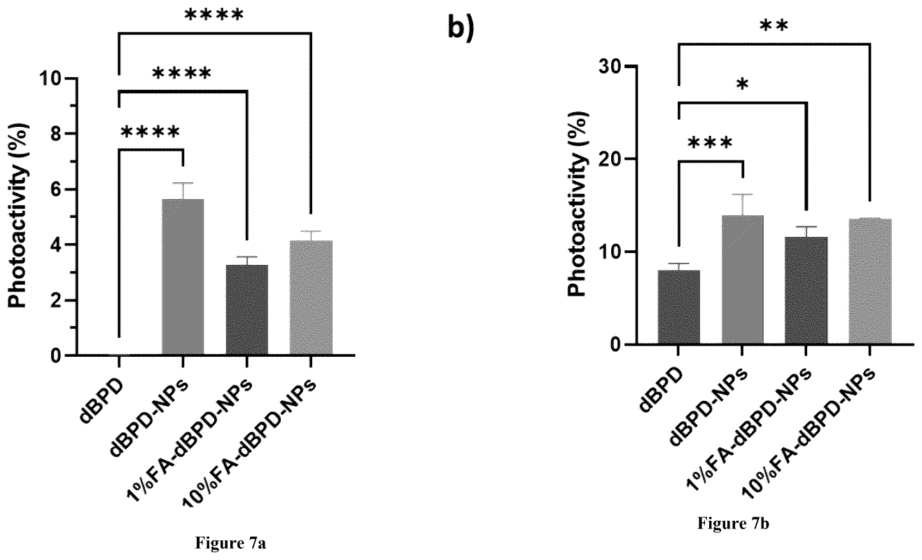

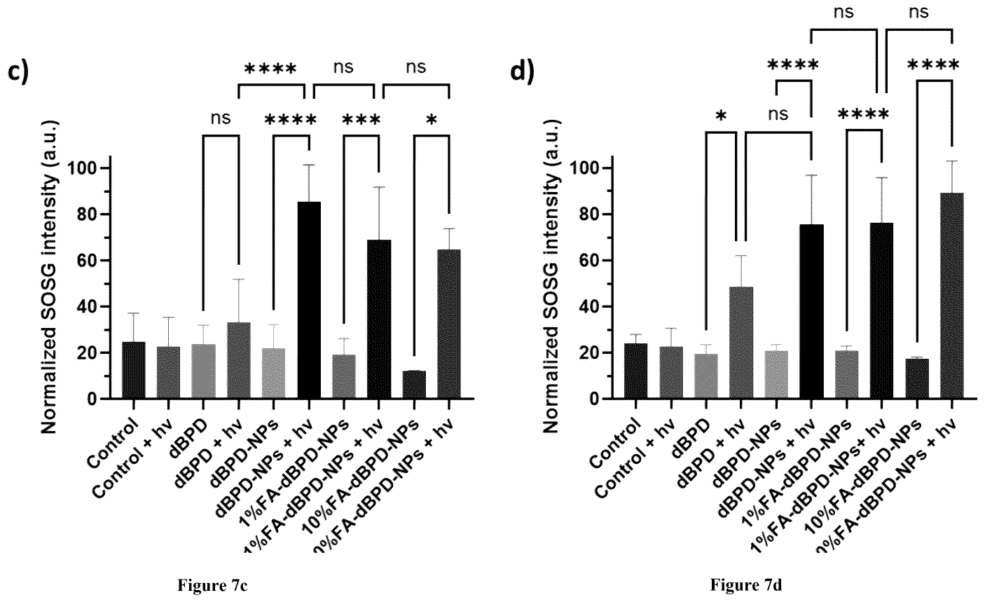

To calculate the photoactivity values for the different drugs and nanodrugs, the maximum fluorescence emission intensity of dBPD in aqueous media was divided by the maximum fluorescence emission intensity of dBPD in DMSO for each formulation (λ_{exc}=435 nm/λₑₘ=689 nm). The photoactivity of the formulations was studied in PBS (FIG. 7a) and human serum 1% (FIG. 7b). The encapsulation of the dBPD within the NPs ensured a significant enhancement of the photoactivity values in both solvents compared to the free dBPD. The enhanced solubility of the dBPD when entrapped in the PEGylated NPs increases the fluorescence emission signal and thus the photoactivity of the drug. All samples showed also higher photoactivity values in 1% human serum albumin (HSA) than in PBS. This could be explained by the rapid interaction of the porphyrin-based photosensitizer with HSA. Next, the singlet oxygen generation was quantified using singlet oxygen sensor green (SOSG) probe in PBS and 1% HSA. All the formulations (2 μM) were prepared in PBS and 1% HSA and kept at 37° C. for 24 h. After this time, the samples were mixed with the SOSG probe and the fluorescence emission was recorded (λ_{exc}=504 nm/λₑₘ=525 nm) before and after excitation at 689 nm (40 J/cm², 150 mW/cm²). The results of the formulations in PBS (FIG. 7c) showed singlet oxygen production for all the nanoparticles and negligible for dBPD. The calculated fold changes between the SOSG signals before and after irradiation were 0, 10, 63, 50 and 53 for no drug, dBPD, dBPD-NPs, 1% FA-dBPD-NPs and 10% FA-dBPD-NPs, respectively. When the samples were dispersed in 1% HSA, the singlet oxygen production was significant for all the drugs including the free dBPD (FIG. 7d). However, the fold change was 2 times higher for the dBPD-NPs, 1% FA-dBPD-NPs and 10% FA-dBPD-NPs compared to dBPD. These results demonstrate that the formation of LNPs with dBPD enhanced the photoactivity performance of the dBPD and thus the singlet oxygen generation in solution, which could be translated to a potential enhancement of the photodynamic therapy efficiency in vitro and in vivo.

### Example 6. Intracellular Uptake and Colocalization of dBPD-NPs

The cytotoxicity of dBPD and dBPD-NPs on ovarian cancer cells (OVCAR8) in the dark is shown in FIG. 19a. A broad range of concentrations (0-15 μM) were evaluated showing enhanced cell viability for dBPD-NPs over dBPD at high concentrations.

The quantitative assessment of intracellular uptake was performed in OVCAR8 and 3T3 cells. Cells were seeded at 20,000 cells/well in 35 mm Petri dishes and allowed to grow overnight. Cells were treated with dBPD, dBPD-NPs, 1% FA-dBPD-NPs and 10% FA-dBPD-NPs (2.5 μM) for 24 h. Subsequently, cells were washed twice with PBS and lysates were collected in radioimmunoprecipitation assay (RIPA) buffer. The concentration of dBPD in cells was determined by dBPD fluorescence measurements using a multi-mode microplate reader (Excitation/Emission: 435/700±10 nm, Synergy Neo2, BioTek) and the appropriate dBPD standard curves. Intracellular dBPD concentration was normalized to total cell protein. The total protein amount was determined using the Pierce BCA protein assay (Thermo Fisher).

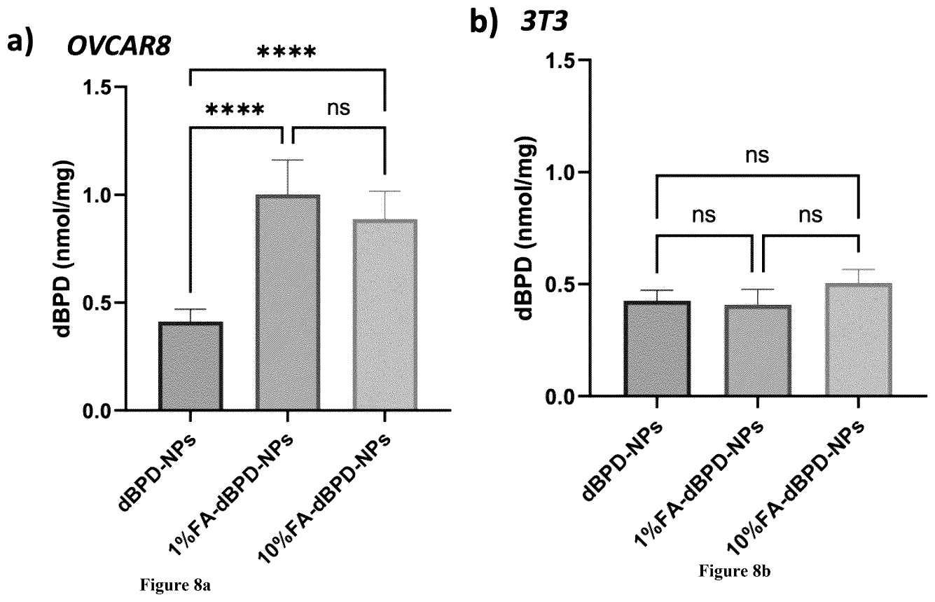

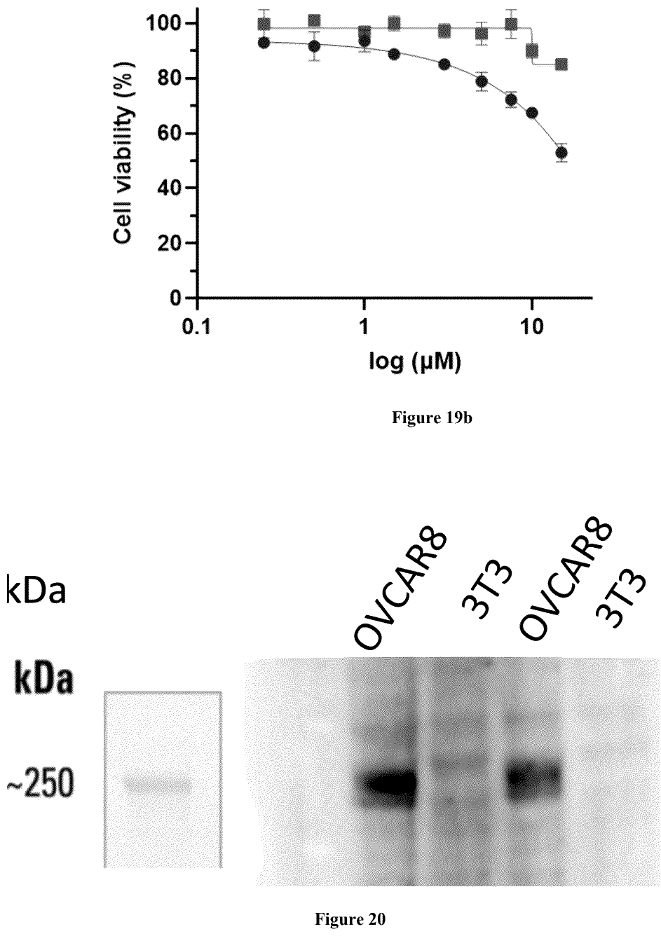

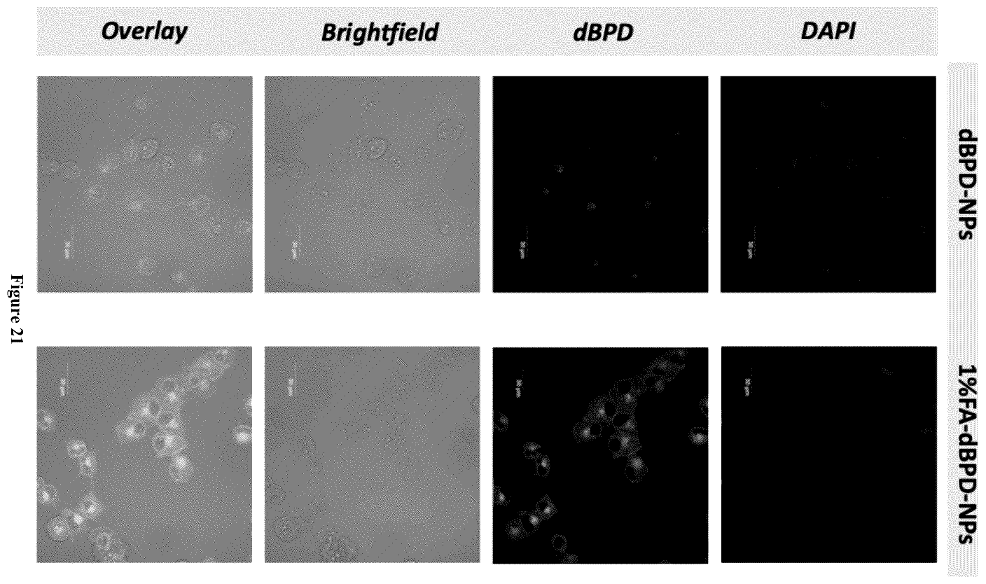

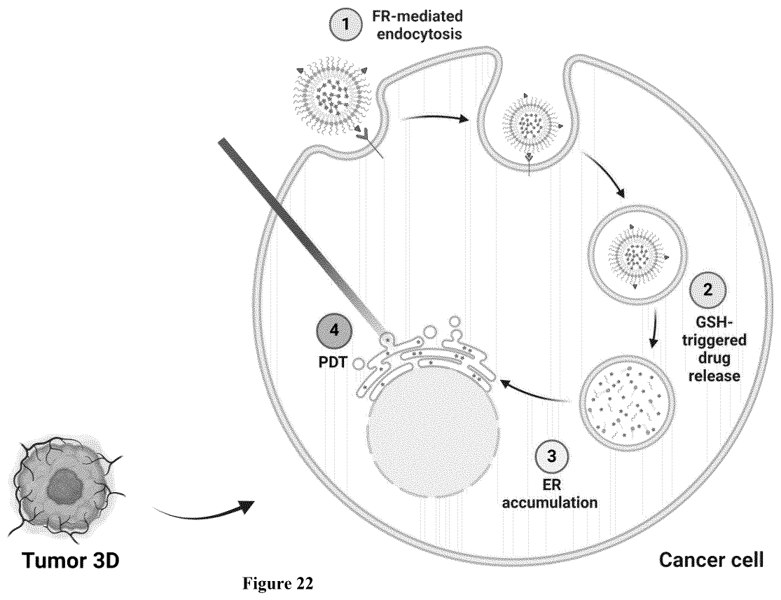

Next, the cellular uptake of the dBPD-NPs was investigated with and without folic acid to determine the ability of the FA-conjugated NPs to enhance the uptake in FR-overexpressing cells. To this end, uptake experiments were performed comparing OVCAR8 (FR-positive cell line) and 3T3 (FR-negative cell line). The OVCAR8 and 3T3 cell lines were confirmed to be FR-positive and negative, respectively, using western blot analysis for FR-α detection with full methods outlined in supplementary information (FIG. 20). Both cell lines were incubated with dBPD-NPs, 1% FA-dBPD-NPs and 10% FA-dBPD-NPs (2.5 UM, 24 h) and then the internalized dBPD was quantified calculating the nmol of dBPD by mg of protein (FIG. 8a and FIG. 8b). The uptake of 1% FA-dBPD-NPs and 10% FA-dBPD-NPs by OVCAR8 cells was significantly (P<0.0001) greater than dBPD-NPs uptake in the same cell line. In contrast, the 3T3 cells showed no significant difference between the uptake of the three nanoformulations tested. Also, the uptake by 3T3 cells was similar to that of dBPD-NPs in OVCAR8 cells and much lower than the targeted formulations in OVCAR8. These results further demonstrated the FR-enhanced uptake of those dBPD-NPs conjugated with FA. However, there was no significant difference in the uptake between 1% FA-dBPD-NPs and 10% FA-dBPD-NPs indicating that the concentration of 1% FA is sufficient to effectively target the FR-positive cells. Therefore, the formulation 1% FA-dBPD-NPs was chosen for all subsequent in vitro and in vivo experiments.

To further explore the targeting of FR overexpressing cells, OVCAR8 cells (FR-positive) were treated with dBPD-NPs and 1% FA-dBPD-NPs (2.5 μM, 24 h) and stained with Hoechst. Fixed cells were imaged in the confocal scanning microscope showing a higher fluorescence emission intensity from the cells treated with 1% FA-dBPD-NPs compared to those treated with dBPD-NPs (FIG. 8c). The conjugation of the NPs with FA increased the uptake of the NPs in OVCAR8 cells further demonstrating the targeting ability of the 1% FA-dBPD-NPs.

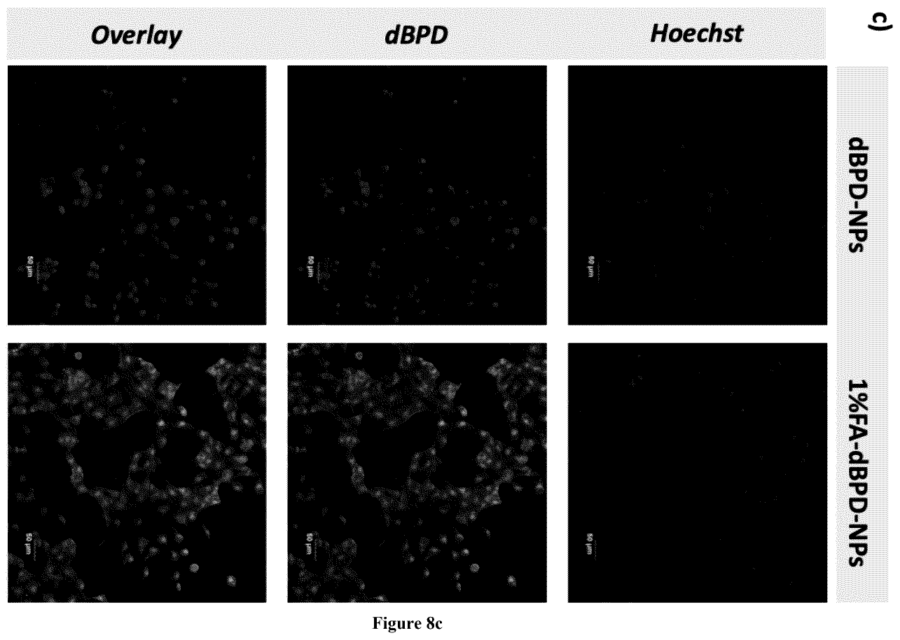

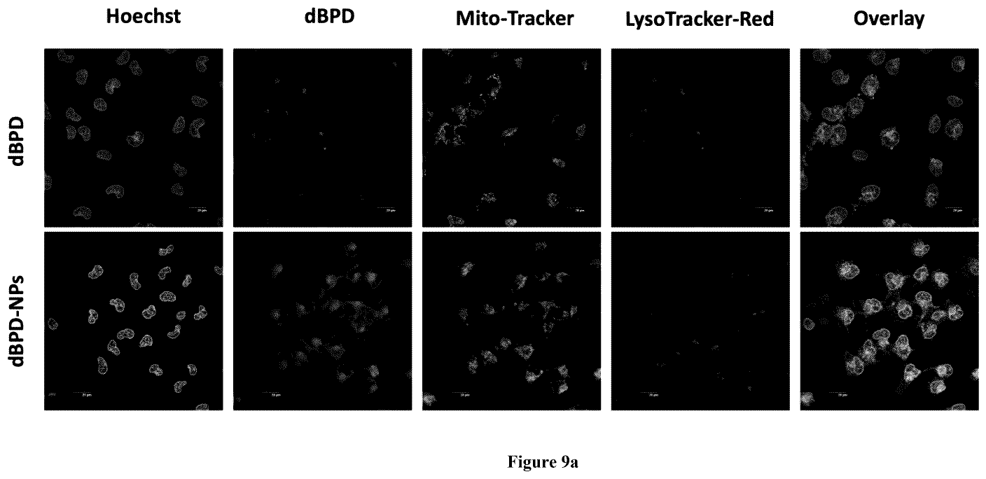

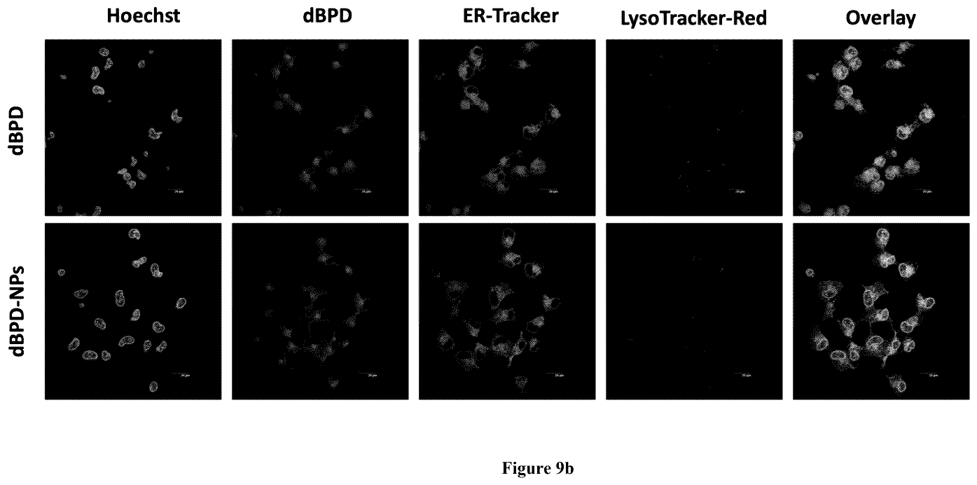

It has been previously demonstrated that free BPD preferably accumulates in the mitochondria. To investigate where the dBPD is accumulated within the cells, colocalization studies were performed in OVCAR8 cells labeling different cellular organelles: mitochondria, lysosomes, nucleus and endoplasmic reticulum (ER). Cells incubated with dBPD (2.5 μM, 24 h) and stained with Lysotracker-Red Mitotracker-Green and confocal scanning microscopy images were acquired and analyzed for subcellular colocalization. The intensity plots for representative cells (FIG. 9) showed the accumulation of the drug in the lysosomes of the cells with a Pearson's coefficient of 0.61. The colocalization of the dBPD with the mitotracker showed a lower Pearson's coefficient (0.47) indicating the decreased accumulation of the drug in these organelles. Cells incubated with dBPD were also stained with ER-tracker and the results showed a good colocalization (Pearson's coefficient of 0.65).

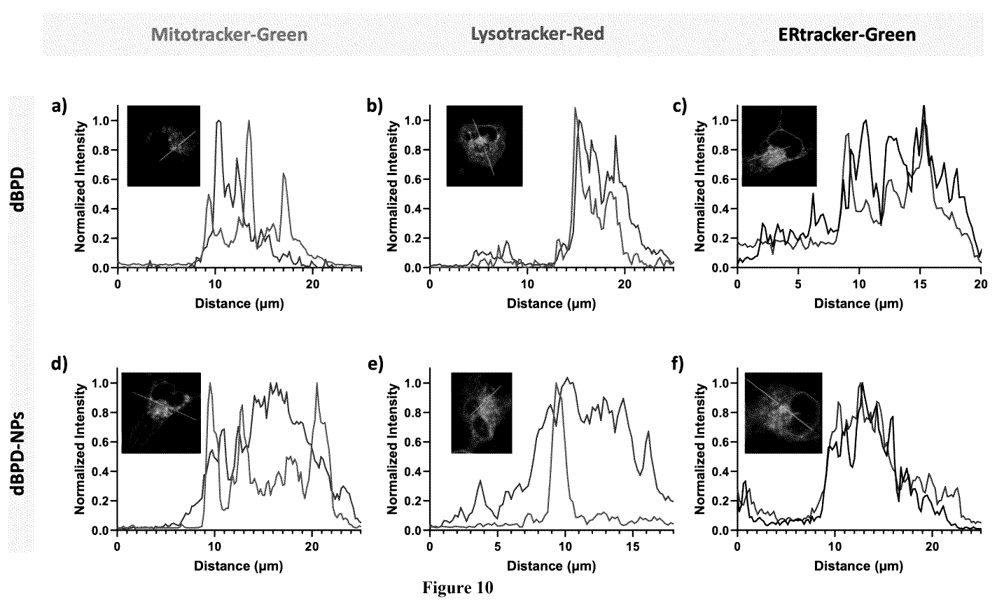

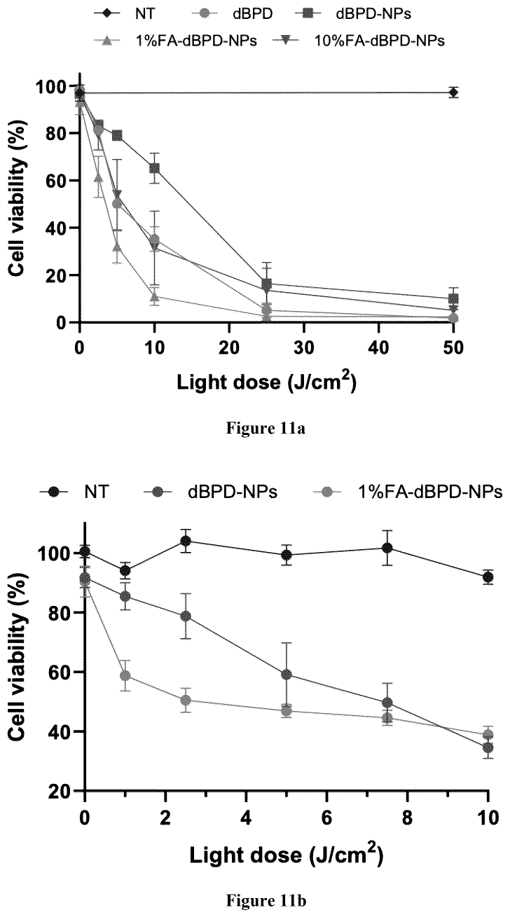

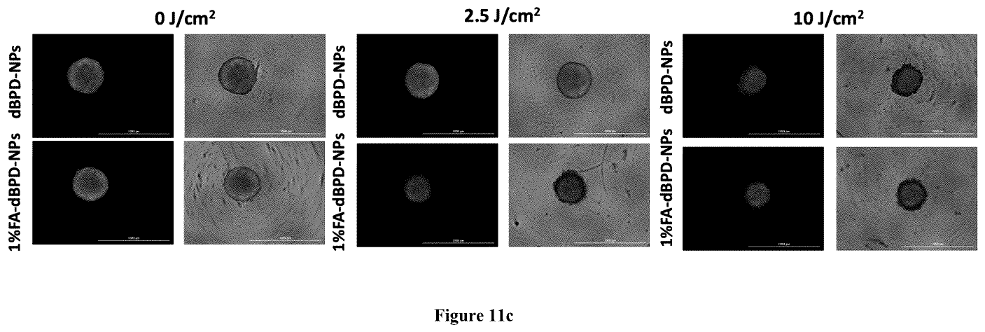

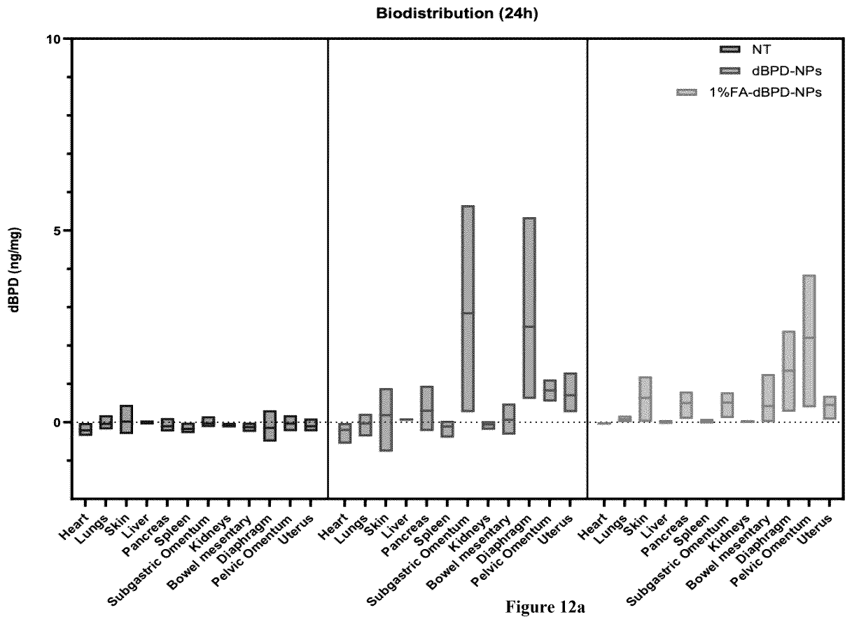

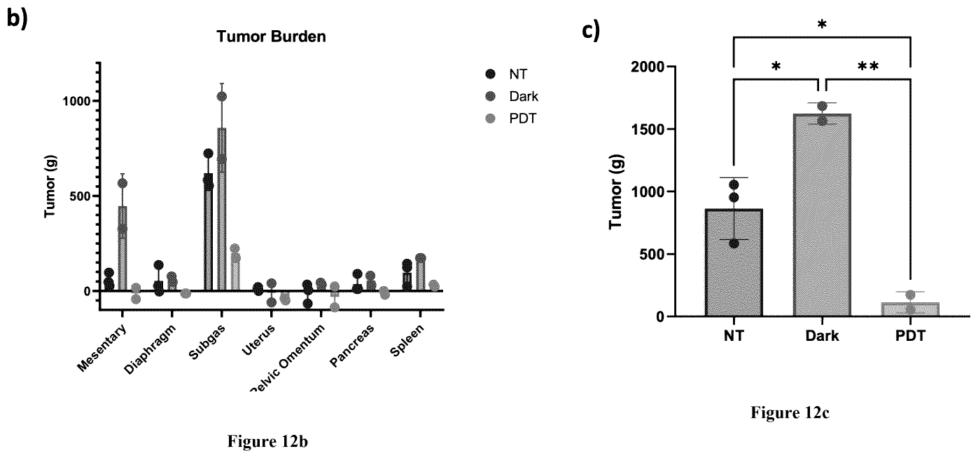

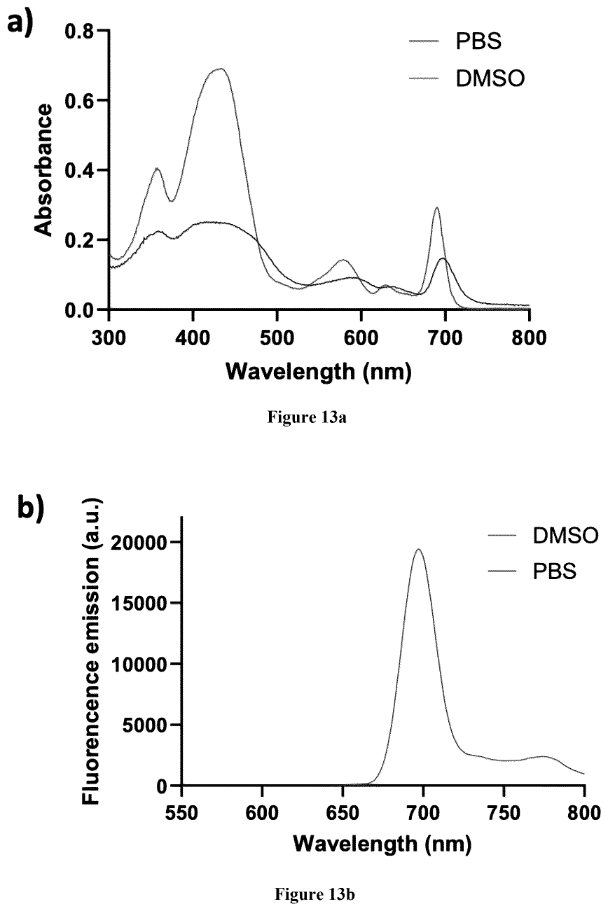

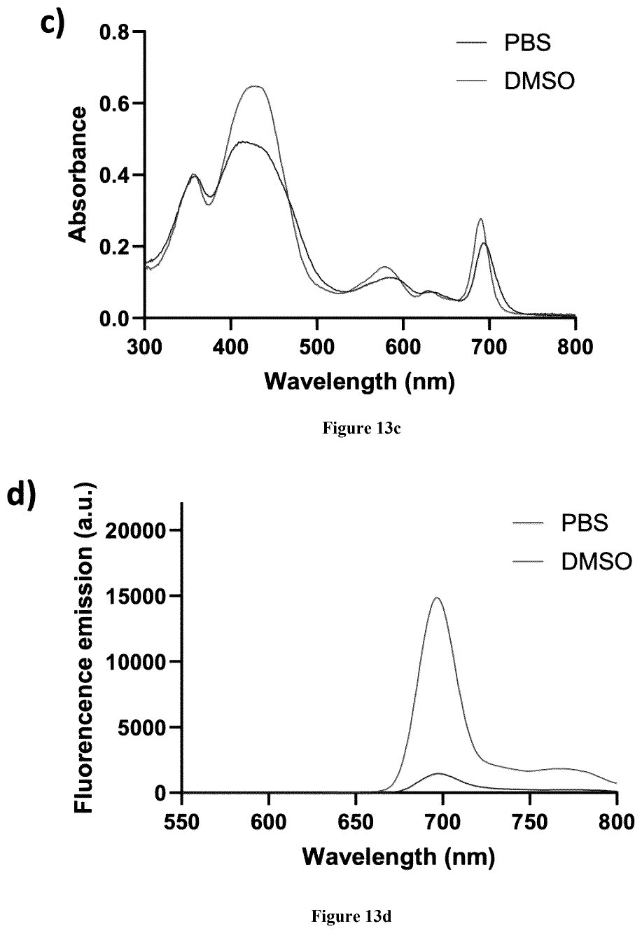

Cells incubated with 1% FA-dBPD-NPs (2.5 μM, 24 h) and stained with MitoTracker Green FM and Lysotracker Red DND-99. Confocal microscopy images showed poor colocalization of the 1% FA-dBPD-NPs in the mitochondria and a higher preference for the lysosomes (FIG. 9a). These results confirmed the different distribution of the dBPD within the cells compared to BPD. Even though dBPD showed a partial accumulation into lysosomes, the fluorescence emission signal was mainly localized in other organelle of the cells. The endoplasmic reticulum (ER) membrane is a lipid-soluble system which can trap lipid-soluble molecules. Based on the high hydrophobicity and excellent lipophilicity of the drug, it was hypothesize that the dBPD could potentially be accumulated in the ER of the cells. To demonstrate this, cells were incubated with dBPD, dBPD-NPs and 1% FA-dBPD-NPs (2.5 μM, 24 h) and stained with Hoechst, ER-Tracker™ Green and Lysotracker Red DND-99. Confocal scanning microscopy images of live cells showed the accumulation of the dBPD in the ER for all the formulations tested (FIG. 9b). The fluorescence intensity graphs where plotted for representative cells to further determine the colocalization results (FIG. 10).

### Example 7. Photodynamic Therapy Effect of Ovarian Cancer Cells: 2D and 3D Cancer Cell Culture

To investigate the photodynamic therapy effect of the dBPD, dBPD-NPs and 1% FA-dBPD-NPs in ovarian cancer cells, 2D and 3D cell cultures of OVCAR8 were prepared. 2D cultured cells were treated with a fixed concentration of the drug (0.25 μM) and irradiated at different light doses. The killing curves obtained for OVCAR8 at different light doses showed superior treatment efficiency for 1% FA-dBPD-NPs compared to the other formulations tested. Interestingly, the 1% FA-dBPD-NPs not only enhanced the effect of the dBPD-NPs but also that of the free dBPD (FIG. 11a). The difference between dBPD-NPs and 1% FA-dBPD-NPs or 10% FA-dBPD-NPs can be explained by the higher uptake of dBPD when the nanoformulation is modified with FA, as demonstrated previously. To determine the PDT efficiency of the dBPD-based formulations, the cellular uptake of concentration of dBPD calculated previously was correlated with the PDT-mediated viability reduction (Table 1). As expected, the PDT efficiency of 1% FA-dBPD-NPs was found to be significantly higher than that of free dBPD as calculated at two different light doses (2.5 and 10 J/cm²). The dBPD-NPs and 10% FA-dBPD-NPs showed a higher PDT efficiency but with no significant difference compared to dBPD. These results suggest that the 1% FA-dBPD-NPs is the optimal nanoformulation of dBPD for the PDT of OVCAR8.

TABLE 1
Photodynamic therapy (PDT) efficiency. PDT efficieny
of free dBPD, dBPD-NPs, 1% FA-dBPD-NPs and 10% FA-dBPD-
NPs in OVCAR8 cells. PDT efficiency is calculated as the percentage
of viability reduction per fmole of intracellular dBPD. PDT
with irradiance of 150 mW/cm².

PDT efficiency
690 nm light dose (J/cm²)

| Groups | 2.5 | 10 |
|---|---|---|
| dBPD | 0.12 ± 0.02% | 0.41 ± 0.04% |
| dBPD-NPs | 0.29 ± 0.04% | 0.48 ± 0.06% |
| 1% FA-dBPD-NPs | 0.38 ± 0.08%** | 0.97 ± 0.18%* |
| 10% FA-dBPD-NPs | 0.23 ± 0.14% | 0.86 ± 0.36% |

*p ≤ 0.05
**p ≤ 0.01

Next, a three-dimensional culture formed by OVCAR8-DsRed2 cells was used for the PDT, to further demonstrate the selectivity of the 1% FA-dBPD-NPs and the penetration of the drug in a better mimicking tumor structure. The spheroids were cultured following a 3D ovarian cancer culture model. OVCAR8-DsRed2 cells were seeded in ultra-low attachment round bottom plates. Spheroids were fully established by day 3 and were treated with dBPD, dBPD-NPs or 1% FA-dBPD-NPs at a fixed concentration of the drug (0.25 μM, 24 h). After this time, spheroids were irradiated at different light doses and cell viability was evaluated 48 h after PDT. Images were taken longitudinally on days 1, 3, 5 and 7 from seeding. On day 7, the cell viability was determined by (a) measuring the fluorescence emission intensity of each spheroid and normalized to the untreated spheroid; and (b) CellTiter-Glo® Cell Viability Assay. Viability curves showed the superior PDT performance of the 1% FA-dBPD-NPs over dBPD-NPs at low light doses (FIG. 11b). These results proved the selectivity of the 1% FA-dBPD-NPs towards the OVCAR8 cells and, therefore, its superior performance as a photosensitizer. Representative images of the spheroids treated with the corresponding dBPD formulation and at different light doses showed the different treatment outcome at 2.5 J/cm² between the dBPD-NPs and the 1% FA-dBPD-NPs demonstrating the higher PDT efficiency of the 1% FA-dBPD-NPs at lower light doses (FIG. 11c).

High-grade serous ovarian cancer cell lines OVCAR8-DsRed2 were obtained. Cells were cultured in RPMI-1640 medium (Corning) supplemented with 10% fetal bovine serum (Gibco), 100 U/mL penicillin and 100 μg/mL streptomycin (Corning). In every four passages, media was supplemented with G418 (Invitrogen) at 500 μg/mL OVCAR8-DsRed2. 3D spheroidal cultures were generated by plating 1000 cells per well in ultra-low-attachment, round bottom 96 well plates (PerkinElmer). The Lionheart FX Automated Microscope (Biotek) was used for imaging 24 hours after plating, and then every two days up to day 7. For treatment evaluation, 2,000 cells (1000 of each cell line) were treated on day 3 for 24 hours before light activation (690 nm, Modulight, Inc.) on day 4. Treatments were done with dBPD, dBPD-NPs or 1% FA-dBPD-NPs (0.25 M) and light activation was performed at 0, 1, 2.5, 5, 7.5 and 10 J/cm² (150 mW/cm²). Longitudinal imaging was conducted as described above, and final cell viability analysis was conducted on day 7 using the CellTiter-Glo® Cell Viability Assay (Promega).

TABLE 2
Comparison of the chemical/physical and physiological
properties of dimeric BPD nanoparticles (dBPD-NPs)

| | | | | |
|---|---|---|---|---|
| Properties | Visudyne | Lipid- | LNP | Folate Receptor- |
| | (Clinical | Anchored | BPD-PC² | Targeted Dimeric |
| | Formulation)¹ | BPD¹ | | BPD Nanoparticles |
| | | | | (dBPD-NPs)⁴ |
| Description | BPD - liposomal | A custom lipid- | A solid lipid | Dimeric BPD |
| | encapsulation. | anchored BPD | nanoparticle | (dBPD) - two BPD |
| | Each mL of | liposome | (LNP) | molecules |
| | reconstituted | consisting of | formulated | connected via a |
| | Visudyne contains | DPPC, DOTAP, | from DPPC, | disulfide linker, |
| | 2 mg BPD | DSPE-PEG2000, | DMG-PEG, | enabling self- |
| | (verteporfin); | and BPD | cholesterol, | assembly into lipid |
| | inactive | covalently linked | SM-102, and | nanoparticles. |
| | ingredients include | to 16:0 lyso- | BPD-PC (BPD | Functionalized with |
| | ascorbyl palmitate, | phosphatidylcholine. | conjugated to | folic acid (FA) for |
| | BHT, dimyristoyl | | 20:0 lyso-PC). | targeting, and |
| | phosphatidylcholine, | | | disulfide bridges |
| | egg | | | allow GSH- |
| | phosphatidylglycerol, | | | triggered release in |
| | lactose. | | | high-GSH tumor |
| | | | | environment. |
| Formulation | Liposome | Liposome | Lipid | Cancer targeted |
| type | | | nanoparticle | lipid nanoparticle |
| | | | (LNP) | (LNP) |
| Molecular | 718.8 | 1194.8 | 1224.5 | 1553.6 |
| weight of | | | | |
| BPD variant | | | | |
| Nanoparticle | 542.8 +/− 547.5 nm | 106.1 +/− 0.515 nm | 158 +/− 1 nm | 117 +/− 5 nm |
| size | (reconstituted; | | | |
| | highly variable | | | |
| | upon dilution) | | | |
| Polydispersity | 0.877 +/− 0.217 | 0.063 +/− 0.001 | 0.09 +/− 0.02 | 0.15 +/− 0.05 |
| | High (broad | Low | Low | Low |
| | distribution) | (monodispersed) | (monodispersed) | (monodispersed) |
| BPD | 485 ± 75³ | No reported | No reported | 218.60 ± 20.72 |
| molecules | | | | |
| per | | | | |
| nanoparticle | | | | |
| Targeting | None | None | None | Active folate |
| mechanism | | | | receptor targeting |
| | | | | via folic acid |
| Release | Passive | Passive | Passive | Triggered release |
| mechanism | | | | via GSH-cleavable |
| | | | | disulfide bonds |
| Intracellular | Non-specific | Lysosomes | No reported | High accumulation |
| accumulation | (primary | | | in FR+ cells; |
| | mitochondria); | | | colocalization with |
| | relies on passive | | | endoplasmic |
| | diffusion | | | reticulum |

### Example 8. Cell Culture

High-grade serous ovarian cancer cell line OVCAR8 and mouse 3T3 fibroblast cells were obtained from ATCC and cultured per the vendor's instructions. The OVCAR8 cell line was cultured in RPMI-1640 medium (Corning) supplemented with 10% fetal bovine serum (Gibco), 100 U/mL penicillin and 100 μg/mL streptomycin (Corning). 3T3 cell line was cultured in Dulbecco's Modified Eagle Medium (DMEM, Corning) supplemented with 10% fetal bovine serum (Gibco), 100 U/mL penicillin and 100 μg/mL streptomycin (Corning). Cells were maintained in 5% CO₂ at 37° C. and tested to be mycoplasma-free (MycoAlert, Lonza).

### Example 9. Western Blot Method for FR-Alpha

Primary antibodies used were folate receptor alpha monoclonal antibody (548908) and beta-actin monoclonal antibody (8H10D10) #3700. The secondary antibody used was anti-mouse IgG, HRP-linked antibody #7076. Untreated OVCAR8, OVCAR8-DsRed2, or 3T3 cell lysates were collected in RIPA buffer supplemented with a 1% protease and phosphatase inhibitor cocktail (ThermoFisher, USA). Western blotting was performed as previously described (PMID: 31856423), with 10 μg of cell lysate loaded per lane. Proteins were detected using antibodies against folate receptor alpha (1:1000, ThermoFisher 548908) and beta-actin (1:5000, Cell Signaling #3700) and membranes were imaged using Ultimate Near-infrared Fluorescent Imager Azure 500 (Azure Biosystems).

### Example 10. Cytotoxicity Assay

CellTiter-Glo® Cell Viability Assay (Promega) was used to evaluate the in vitro cytotoxicity of dBPD and dBPD-NPs against OVCAR 8 and NCI/ADR-Res cell lines. Cells (2000 cells/well) were seeded in a 96-well plate and incubated at 37° C. and 5% CO₂ for 48 h. Next, cells were treated with serial concentrations of the dBPD or dBPD-NPs (0, 0.25, 0.5, 1.0, 1.5, 3.0, 5.0, 7.5, 10 and 15 μM) for 48 h. At the end of the incubation period, each well was evaluated with the CellTiter-Glo® Cell Viability Assay.

### Example 11. Confocal Microscopy

Uptake and colocalization studies were performed using a FV3000 Confocal Laser Scanning Microscope (Olympus). OVCAR8 cells were plated in a glass 24-well plate (Sensoplate, PS, F-Bottom, glass bottom, black well, Greiner Bio-One) at 20,000 cells/well and incubated for 24 h at 37° C. and 5% CO₂. Cells were treated with dBPD, dBPD-NPs or 1% FA-dBPD-NPs (2.5 μM) for 24 h. After this time, cells were washed twice with PBS. For uptake images of dBPD-NPs vs 1% FA-dBPD-NPs, cells were labelled with Hoechst (NucBlue™ Live Cell Stain ReadyProbes™ reagent, Invitrogen, 2 drops/mL, 10 min). For colocalization studies, cells were treated with Hoechst (2 drops/mL, 10 min), LysoTracker™ Red DND-99 (Invitrogen, 100 nM, 1 h) and Mitotracker™ Green FM (Invitrogen, 100 nM, 30 min) or ER-Tracker™ Green (BODIPY™ FL glibenclamide, Invitrogen, 1 μM, 30 min). After 3 washes with PBS, 1 mL of PBS was added to each well and life cells were directly imaged in the confocal microscope unless otherwise stated. Channels used in the confocal microscope were: Hoechst (λ_{exc}=405 nm, λₑₘ=430-470 nm), Mitotracker Green (λ_{exc}=488 nm, λₑₘ=500-535 nm), ER-Tracker Green (λ_{exc}=488 nm, λₑₘ=500-535 nm), LysoTracker Red (λ_{exc}=561 nm, λₑₘ=565-620 nm), dBPD (λ_{exc}=445 nm, λₑₘ=650-750 nm) and BF (λ_{exc}=445 nm). Images were analyzed using CellSens FV software and Image J.

### Example 12. Statistical Analysis

GraphPad Prism version 9.0.2 was used for statistical analysis. All data shown were collected at least in triplicate and plotted as mean±standard error of the mean. Details regarding statistical testing are elaborated in figure captions, and statistical significance was determined as P<0.05.

All publications mentioned herein are incorporated by reference to the extent they support the present invention.

### REFERENCES

1. Obaid, G., Celli, J. P., Broekgaarden, M. et al. Engineering photodynamics for treatment, priming and imaging. Nat Rev Bioeng (2024). https://doi.org/10.1038/s44222-024-00196-z
2. Birrer, M. J., Betella, I., Martin, L. P., & Moore, K. N. (2019). Is Targeting the Folate Receptor in Ovarian Cancer Coming of Age?. The oncologist, 24(4), 425-429. https://doi.org/10.1634/theoncologist.2018-0459
3. Chizhu Ding, Zibiao Li, A review of drug release mechanisms from nanocarrier systems, Mat. Sci. Eng. C, 76, 2017, 1440-1453. https://doi.org/10.1016/j.msec.2017.03.130.
4. Rizvi I, Nath S, Obaid G, Ruhi M K, Moore K, Bano S, Kessel D, Hasan T. A Combination of Visudyne and a Lipid-anchored Liposomal Formulation of Benzoporphyrin Derivative Enhances Photodynamic Therapy Efficacy in a 3D Model for Ovarian Cancer. Photochem Photobiol. 2019 January; 95 (1): 419-429. doi: 10.1111/php.13066.
5. Shah N, Soma S R, Quaye M B, Mahmoud D, Ahmed S, Malkoochi A, Obaid G. A Physiochemical, In Vitro, and In Vivo Comparative Analysis of Verteporfin-Lipid Conjugate Formulations: Solid Lipid Nanoparticles and Liposomes. ACS Appl Bio Mater. 2024 Jul. 15; 7 (7): 4427-4441. doi: 10.1021/acsabm.4c00316.
6. Quinlan J A, Inglut C T, Srivastava P, et al. Carrier-free, amorphous verteporfin nanodrug for enhanced photodynamic cancer therapy and brain drug delivery. Adv Sci. 2024; 11 (17): 2302872. doi: 10.1002/advs.202302872
7. Arnau del Valle C, Srivastava P, McNaughton K, Huang H-C. Self-assembly of verteporfin dimers into folate receptor-targeted lipid nanoparticles for photodynamic therapy of ovarian cancer. Bioeng Transl Med. 2025; e70031. doi: 10.1002/btm2.70031

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

1. A dimeric benzoporphyrin derivative (dBPD) of Formula I:

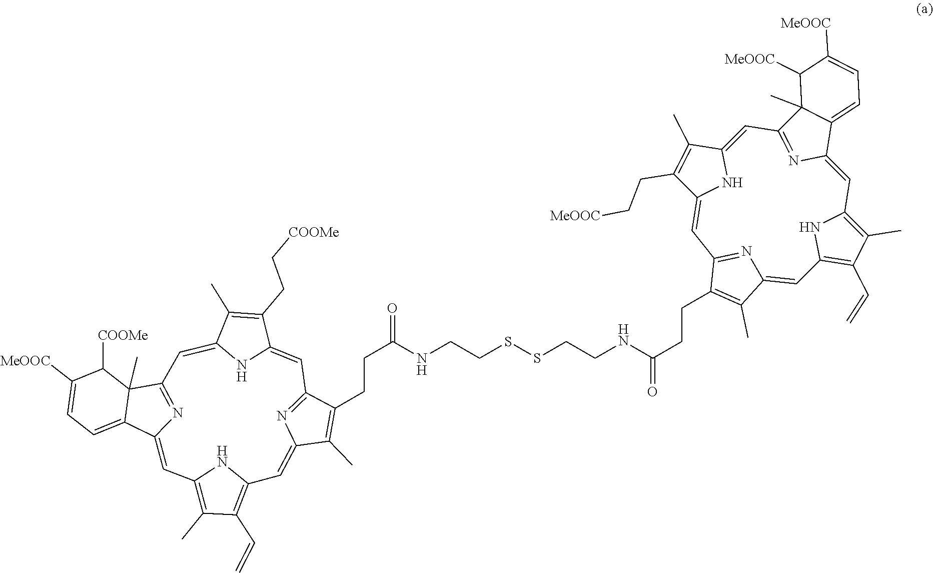 [SMILES: C=Cc1c(C)c2cc3nc(cc4[nH]c(cc5nc(cc1[nH]2)C(C)=C5CCC(=O)NCCSSCCNC(=O)CCC1=C(C)c2cc5[nH]c(cc6nc(cc7[nH]c(cc1n2)c(CCC(=O)OC)c7C)C1(C)C6=CC=C(C(=O)OC)C1C(=O)OC)c(C)c5C=C)c(CCC(=O)OC)c4C)C1(C)C3=CC=C(C(=O)OC)C1C(=O)OC]

2. The dimeric molecule of claim 1, wherein said dBPD is formed by conjugating two benzoporphyrin derivative molecules using cystamine as the linker moiety by EDC/HOBt coupling reaction between the amine groups in the cystamine and the carboxyl group on BPD.

3. A nanoparticle composition, said nanoparticle composition comprising a dBPD and polyethylene glycol (PEG), wherein said PEG is chosen from, DSPE-mPEG2000, DSPE-mPEG1000, DSPE-mPEG750, DSPE-mPEG5000, Cholesterol-mPEG2000, mPEG-DSG, mPEG-DMG, mPEG-PLGA, and mPEG-PLA.

4. The nanoparticle composition of claim 3, wherein said PEG is arranged in the form of a liposome and said dBPD is encapsulated within said nanoparticle.

5. The nanoparticle composition of claim 3, wherein said nanoparticle composition is monodispersed.

6. The nanoparticle composition of claim 3, wherein said nanoparticle composition further comprises folic acid.

7. The nanoparticle composition of claim 6, wherein said folic acid is tethered to said PEG on the outer portion of said capsule.

8. The nanoparticle composition of claim 7, wherein said folic acid is tethered to said PEG and is present on at least 50% or more of the surface of said nanoparticle.

9. The nanoparticle composition of claim 3, wherein said nanoparticle composition is photoactive.

10. The nanoparticle composition of claim 9, wherein said photoactivated nanoparticle is selectively cytotoxic to cancer cells only.

11. A method of making a dBPD-loaded nanoparticle, said method comprising:
a. dissolving dBPD in an organic polar solvent;
b. mixing solution of step a with a pegylated lipid;
c. evaporating said organic solvent from the mixture of step b to form a solid;
d. hydrating the solid with an aqueous solution; and
e. freeze-thawing the solution of step d to form dBPD-NPs.

12. The method of claim 11, wherein said solvent is chosen from chloroform, methanol, ethanol, acetone, dichloromethane, and tetrahydrofuran.

13. (canceled)

14. (canceled)

15. A method of treating cancer, said method comprising administering a pharmaceutically effective amount of dBPD or a formulation comprising a nanoparticle composition and one or more pharmaceutically acceptable excipients to a patient or subject in need thereof;
wherein said nanoparticle composition comprises a dBPD and polyethylene glycol (PEG).

16. The method of claim 15, wherein said formulation releases dBPD from the dBPD-loaded nanoparticle in the presence of glutathione (GSH).

17. The method of claim 15, wherein said formulation is cytotoxic when said formulation is photoactivated.

18. The method of claim 17, wherein said photoactivated formulation is selectively cytotoxic to cancer cells only.

19. The method of claim 17, wherein said photoactivated formulation is selectively delivered to the endoplasmic reticulum of said cancer cells.

20. The method of claim 15, wherein said cancer is lung cancer, ovarian cancer, endometrial cancer, or breast cancer.

21. The method of claim 11, wherein said method further comprises adding folic acid to the mixture of step b.
